(19) United States
(12) Patent Application Publication
(10) Pub. No.: US 2012/0177597 A1
(43) Pub. Date: Jul. 12, 2012

(54) COMPOSITIONS AND METHODS OF ALTERATION OF AUTOIMMUNE DISEASES

(75) Inventors: Hideki Ueno, Plano, TX (US); Jacques F. Banchereau, Montclair, NJ (US); Nathalie Schmitt, Dallas, TX (US)

(73) Assignee: BAYLOR RESEARCH INSTITUTE, Dallas, TX (US)

(21) Appl. No.: 13/345,576

(22) Filed: Jan. 6, 2012

Related U.S. Application Data
(60) Provisional application No. 61/431,986, filed on Jan. 12, 2011.
(60) Provisional application No. 61/545,447, filed on Oct. 10, 2011.

(51) Int. Cl. A61K 38/20; A61K 39/00; A61P 37/04; C12N 5/02; A61K 39/395; A61K 35/26; C12N 5/0783

(52) U.S. Cl. 424/85.2; 424/93.71; 424/184.1; 435/325; 435/377; 424/145.1

(57) ABSTRACT

The present invention provides methods and compositions for affecting functional differences of blood memory CD4+ T cell populations in a subject by providing isolated and purified T cell subsets selected from X5⁺ CD4⁺ or X5⁻ CD4⁺ T cells to a subject. Another invention includes a method for regulating CD4+ T cells comprising the steps of isolating and purifying one or more naïve CD4+ T cells from a subject; contacting the one or more naïve CD4+ T cells with one or more cytokines selected from IL-6 and TGF-b, IL-12, or TGF-b and IL-12; and modifying the expression of the factors from the naïve CD4+ T cells wherein the one or more factors are the expression of IL-21, the decreased expression of IFN-g, the expression of CXCR5, the expression of Bcl-6, the decreased expression of Blimp-1, or a combination thereof.

### CROSS-REFERENCE TO RELATED APPLICATIONS

This application claims priority to U.S. Provisional Application Ser. No. 61/431,986, filed Jan. 12, 2011 and U.S. Provisional Application Ser. No. 61/545,447, filed Oct. 10, 2011, the entire contents of each are incorporated herein by reference.

### STATEMENT OF FEDERALLY FUNDED RESEARCH

This invention was made with U.S. Government support under Contract Nos. U19-AI057234, R01-CA84512, R01-CA078846, AR054083-01 awarded by the National Institutes of Health (NIH). The government has certain rights in this invention.

### TECHNICAL FIELD OF THE INVENTION

The present invention relates in general to the field of antigen presentation and immune cell activation, and more particularly, to the activation of immune cells through the CXCR5 (X5)⁺ CD4⁺, and the treatment of, e.g., dermatomyositis, and to compositions and methods for the treatment of autoimmune diseases by blocking the development of T follicular helper (Tfh) cells.

### BACKGROUND OF THE INVENTION

Without limiting the scope of the invention, its background is described in connection with activation of immune cells.

Antibody response is largely dependent on the help provided by CD4⁺ T cells. Early in vitro studies using clones and recombinant cytokines have concluded that multiple CD4⁺ T cell subsets contribute to the regulation of antibody response. T follicular helper (Tfh) cells represent a subset of CD4+ T cells, which express chemokine (C-X-C motif) receptor 5 (CXCR5) which allows their migration into follicles in response to the specific ligand, CXCL13 secreted by follicular dendritic cells. IFN-γ secreted by T helper (Th)1 cells induces mouse B cell class-switching towards IgG2a, while IL-4 and IL-13 secreted by Th2 cells promote class-switching towards IgG1 and IgE. CD4⁺ T cells are fundamental for the generation of germinal centers GCsCD4⁺ T cells present in B cell follicles, have been recently established as a Th subset specialized for providing help to B cells in GCs and which are discrete structure in secondary lymphoid organs where selection of high-affinity B cells and development of B cell memory. Tfh cells secrete IL-4, IL-10 and IL-21, cytokines that efficiently promote growth, differentiation, and class-switching of B cells. Tfh cells also express multiple surface molecules essential for the helper functions, including CD40-ligand (CD40L), and inducible co-stimulator (ICOS). Tfh cells express high levels of B cell lymphoma 6 (Bcl-6), a transcription factor essential for the development of GC B cells.

U.S. Pat. No. 7,820,790 issued to Bakker et al. (2010) which provides methods for preventing, treating, or ameliorating a disease, disorder, or condition in a patient, comprising administering an IL-6 antagonist to the patient, wherein the IL-6 antagonist comprises a polypeptide comprising at least one LDL receptor class A monomer domain that selectively binds to TL-6 and at least one LDL receptor class A monomer domain that selectively binds to IgG. The disease, disorder, or condition being treated by the Bakker invention comprises an inflammatory and/or autoimmune disease, disorder, or condition.

U.S. Patent Application Publication No. 2008/0254137 (Raymond and Reid, 2008) describes the induction of the regulatory cytokines, interferon gamma and IL-27, as a method to treat autoimmune diseases and a method by which such regulatory cytokines can be induced using a detoxified cobra neurotoxin composition. IL-27 suppresses IL-6/TGF-β mediated T cell proliferation of Th17 subset cells and inhibits the production or IL-17 by CD4+ T cells. As a consequence of it's influence on the Th17 subset and IL-17 production, IL-27 provides a potential control mechanism for IL-17 in cases of autoimmune inflammation.

### SUMMARY OF THE INVENTION

The present invention provides a method for enhancing the efficiency of an antibody response to a vaccination in a subject by providing one or more isolated and purified X5 CD4⁺ T cells selected from X3⁻R6⁻, X3⁻R6⁺ to a subject prior to vaccination, wherein the X5 CD4⁺ T cells induce Type 2 cells and Type 17 cells. In one embodiment, the present invention include compositions and methods for treating dermatomyositis by driving the immune response toward T cells that ameliorate the immune response.

The present invention also provides a method for providing protective mucosal immunity in a subject by providing one or more isolated and purified X5 CD4⁺ T cells selected from X3⁻ R6⁺ to a subject prior to vaccination, wherein the X5 CD4⁺ T cells induce Type 17 cells.

The present invention provides a method for inducing the differentiation of naïve B cells towards plasmablasts in a subject by providing one or more isolated and purified X5 CD4⁺ T cells selected from X5 CD4 T cells or X5⁻ CD4⁺ T cells to a subject. The present invention provides an isolated and purified X5 CD4⁺ T cell having one or more isolated and purified X5CD4⁺ T cells selected from Th1 cells, Th2 cells, or Th17 cells, wherein the X5CD4⁺ T cells are X5⁺ or X5⁻. The present invention provides a method for increasing the effectiveness of antigen presentation in a subject by providing one or more isolated and purified X5 CD4⁺ T cells selected from X5⁺ CD4⁺ T cells or X5⁻ CD4⁺ T cells to a subject. The present invention provides a method for the modulation of cytokine secretion by contacting a naïve B cell with one or more CXCR5⁺ CD4⁺ T cell to produce one or more cytokines. The present invention includes a method for affecting functional differences of blood memory CD4+ T cell populations in a subject by providing one or more isolated and purified X5 CD4⁺ T cells selected from X5 CD4⁺ T cells or X5⁻ CD4⁺ T cells to a subject. The present invention includes a method for the modulation of systemic autoimmunity by providing one or more isolated and purified X5 CD4⁺ T cells selected from X5⁺ CD4⁺ T cells or X5⁻ CD4⁺ T cells to a subject.

The present invention includes a pharmaceutical composition for the modulation of systemic autoimmunity including a pharmaceutically acceptable carrier containing a pharmaceutically acceptable amount of one or more isolated and purified X5 CD4⁺ T cell selected from Th1 cells, Th2 cells, or Th17 cells, wherein the X5CD4⁺ T cells are X5⁺ or X5⁻. The present invention includes a method for the treatment of juvenile dermatomyositis including a pharmaceutically acceptable carrier containing a pharmaceutically acceptable amount of one or more isolated and purified X5 CD4⁺ T cell selected from Th1 cells, Th2 cells, or Th17 cells, wherein the X5CD4⁺ T cells are X5⁺ or X5⁻.

The present invention also includes compositions and methods for regulating the differentiation of human naïve CD4+ T cells into T follicular helper cell (Tfh) lineage are disclosed herein. In various embodiments the present invention describes that IL-6 potently drives differentiation of human naïve CD4+ T cells stimulated with IL-12 or the combination of IL-12 and TGF-b towards Tfh lineage, and induce them to express CXCR5, high levels of Bcl-6, and decreased expression of Blimp-1. Therefore, inhibition of IL-6 can be applied to block the development of Tfh cells in humans, provided that overrepresentation of Tfh cells is associated with the development and/or the exacerbation of human autoimmune diseases, inhibition of IL-6 can be applied as a treatment of human autoimmune diseases including dermatomyositis (JD).

The present invention in one embodiment provides a method for promoting development of IL-21 producing T follicular helper cells (Tfh) in a subject from naïve CD4⁺ T cells comprising the steps of: i) providing the one or more naïve CD4⁺ T cells; ii) contacting the one or more naïve CD4⁺ T cells with a cytokine or a cytokine cocktail selected from: IL-6/TGF-b/IL-12; or IL-12; or TGF-b and IL-12; and iii) differentiating the one or more naïve CD4⁺ T cells into the one or more IL-21 producing Tfh cells.

In one aspect the one or more activated CD4+ T cells are CXCR5+ CD4+X3+ R6+ T cells, CXCR5+ CD4+X3−R6− T cells, CXCR5+ CD4+X3−R6+ T cells, or a combination thereof. In another aspect the method comprises the step of activating the one or more Tfh cells with anti-CD3/CD28 mAbs. In yet another aspect the one or more activated CD4+ T cells produce Ig's, GATA3, or a combination thereof. In another aspect the one or more activated CD4+ T cells produce IgM, IgG, IgA, IgE, or a combination thereof.

Another embodiment disclosed herein relates to a method for regulating or suppressing development of one or more Th1 cells by naïve CD4⁺ T cells in a subject comprising the steps of: (i) isolating and purifying the one or more naïve CD4⁺ T cells from subject; (ii) contacting the one or more naïve CD4⁺ T cells with a cytokine cocktail comprising IL-6/TGF-b/IL-12 to suppress the development of one or more Th1 cells by the naïve CD4⁺ T cells, and (iii) modifying the expression of one or more factors from the one or more naïve CD4⁺ T cells. In one aspect of the method hereinabove the method results in a modification of an expression of one or more factors by the naïve CD4⁺ T cells, wherein the modification comprises an increased expression of IL-21, a decreased expression of IFN-g, an increased expression of CXCR5, an increased expression of Bcl-6, a decreased expression of Blimp-1, or a combination thereof.

In yet another embodiment the present invention provides a method for promoting the development of T follicular helper (Tfh) cells from naïve CD4⁺ T cells, modifying expression of one or more factors by the naïve CD4⁺ T cells, or both in a subject comprising the steps of: (a) isolating and purifying one or more naïve CD4⁺ T cells from subject; (b) promoting differentiation of the one or more naïve CD4⁺ T cells to one or more Tfh cells by contacting the one or more naïve CD4⁺ T cells with a cytokine or a cytokine cocktail, wherein the cytokine or cytokine cocktail is selected from: (i) IL-6 and TGF-b and IL-12; or (ii) IL-12; or 9iii) TGF-b and IL-12; and (c) activating the one or more one or more Tfh cells with an anti-CD3/CD28 mAbs to modify the expression of one or more factors, wherein the modification comprises an increase in an expression of IL-21, a decrease in the expression of IFN-g, an increase in the expression of CXCR5, an increase in the expression of Bcl-6, a decrease in the expression of Blimp-1, or a combination thereof.

In one aspect the one or more naïve CD4+ T cells are activated for at least 1, 2, 3, 4, 5, 6, 7, 8, 9, 10, 11, or more days. In another aspect the one or more naïve CD4+ T cells are activated for at least 7 days. In yet another aspect the method further comprises the step of re-stimulation with PMA/ionomycin.

The instant invention further discloses a method for promoting the development of T follicular helper (Tfh) cells in a subject comprising the steps of: i) isolating and purifying one or more naïve CD4⁺ T cells from subject; ii) contacting the one or more naïve CD4⁺ T cells with Bcl-6; and iii) promoting the development of the one or more naïve CD4⁺ T cells into one or more Tfh cells. The method described herein further comprises the step of activating the one or more one or more Tfh cells with anti-CD3/CD28 mAbs.

Another embodiment of the instant invention provides a method for promoting the differentiation of CD4+ T cells into T follicular helper (Tfh) cells in a subject comprising the steps of: i) providing one or more CD4+ T cells; ii) contacting the one or more CD4+ T cells with one or more cytokines selected from IL-6, IL-12 and TGF-b; and iii) differentiating the one or more CD4⁺ T cells into one or more Tfh cells. In one aspect the method further comprises the step of activating the one or more Tfh cells with anti-CD3/CD28 mAbs, wherein the one or more Tfh cells produce a specific antibody response. In another aspect the subject is in need of immunostimulation or an enhanced immune response.

The present invention in one embodiment discloses a method for suppressing development of one or more T follicular helper (Tfh) cells in a subject comprising the steps of: identifying the subject in need of suppression of the immune response by reduction in one or more Tfh cells; and administering a therapeutically effective amount of an IL-6 antagonist, an IL-6 inhibitor, an anti-IL-6 agent, or any combinations thereof to the subject in an amount sufficient to reduce differentiation of CD4+ T cells into one or more Tfh cells. In one aspect of the method hereinabove the IL-6 antagonist, the IL-6 inhibitor, the anti-IL-6 agent, or any combinations thereof comprise lunasin, tocilizumab, sirukumab, Elsilimomab, an anti-IL-6 monoclonal antibody, 20S,21-epoxy-resibufogenin-3-formate (ERBF), or any combinations thereof.

In yet another embodiment the instant invention relates to a method for suppressing the expression of IFN-g in a subject comprising the step of stimulating naïve CD4+ T cells with IL-6/TGF-b/IL-12, wherein the level of IFN-g expressed is less than that stimulated with IL-12 alone or a combination of IL-12/TGF-b. In one aspect the naïve CD4+ T cells express IL-21 but not IFN-g.

Another embodiment of the present invention provides a method for stimulating chemokine receptor expression levels of T follicular helper (Tfh) cells in a subject comprising the steps of: providing one or more naïve CD4+ T cells; contacting the one or more naïve CD4+ T cells with IL-6/TGF-b/IL-12; and increasing the expression of a chemokine receptor in a human Tfh cell, wherein the level of levels of CXCR5 is higher than the level of CXCR5 when stimulated with IL-12 alone and the chemokine receptor plays a central role for their migration into B cell follicles.

In yet another embodiment the present invention provides a method for treating an autoimmune disease in a subject comprising the steps of: identifying the subject in need of treatment against the autoimmune disease and administering to the subject a therapeutically effective amount of a composition comprising an IL-6 antagonist, an IL-6 inhibitor, an anti-IL-6 agent, or any combinations thereof to the subject in an amount sufficient to treat the autoimmune disease, wherein the composition treats the autoimmune disease by reducing a differentiation of CD4+ T cells in the human subject into one or more T follicular helper (Tfh) cells.

In one aspect of the method above the IL-6 antagonist, the IL-6 inhibitor, the anti-IL-6 agent, or any combinations thereof comprise lunasin, tocilizumab, sirukumab, Elsilimomab, an anti-IL-6 monoclonal antibody, 20S,21-epoxy-resibufogenin-3-formate (ERBF), or any combinations thereof. In a specific aspect the autoimmune disease is dermatomyositis.

One embodiment of the present invention relates to an IL-6 activated CD4+ T cell comprising one or more isolated and purified activated CD4+ T cell that expresses IL-21, CXCR5, and Bcl-6 and does not express IFN-g or Blimp-1 as a result of contact with IL-6/IL-12/TGF-b to induce the CD4+ T cells to express IL-21, CXCR5, and Bcl-6 and to acquire the capacity to help B cells.

The present invention in one embodiment relates to a pharmaceutical composition for the modulation of systemic autoimmunity comprising a pharmaceutically acceptable carrier containing a pharmaceutically acceptable amount of one or more isolated and purified activated CD4+ T cell that expresses IL-21, CXCR5, and Bcl-6 and does not express IFN-g or Blimp-1 as a result of contact with IL-6/IL-12/TGF-b to induce the CD4+ T cells to express IL-21, CXCR5, and Bcl-6 and to acquire the capacity to help B cells, wherein the one or more isolated and purified activated CD4+ T cell are CXCR5+CD4+ Th1 T cells, CXCR5+CD4+ Th2 T cells, CXCR5+CD4+ Th17 T cells, CXCR5-CD4+ Th1 T cells, CXCR5−CD4+ Th2 T cells, or CXCR5−CD4+ Th17 T cells.

Another embodiment disclosed herein relates to a method for enhancing the migration of T follicular helper (Tfh) cells into B cell follicles in a subject comprising the steps of: i) identifying the subject in need of enhanced migration of the one or more Tfh cells into B cell follicules; ii) isolating and purifying one or more CD4+ T cells from the subject; iii) activating the one or more CD4+ T cells with IL-6/IL-12/TGF-b to form one or more activated CD4+ T cells that express IL-21, CXCR5 and Bcl-6, and that acquire increased capacity to migrate into B cells to help B cells; and iv) reintroducing the one or more activated CD4+ T cells into the subject.

Another embodiment of the present invention discloses a method for stimulating IgG production in a subject comprising the steps of: isolating one or more T cells from the subject; isolating and purifying one or more CD4⁺ T cells from the one or more T cells; activating the one or more CD4⁺ T cells with IL-6/IL-12/TGF-b to form one or more activated CD4⁺ T cells that express IgG, wherein the levels of IgG is higher than the level of IgG stimulated with IL-12 alone or the combination of IL-12/TGF-b; and reintroducing the one or more activated CD4⁺ T cells into the subject.

In yet another embodiment the present invention provides a method for sending one or more activation signals through STAT3 in a subject comprising the steps of: isolating and purifying one or more CD4⁺ T cells from the subject; activating the one or more CD4⁺ T cells with IL-6/IL-12/TGF-b to form one or more activated CD4⁺ T cells that differentiate into one or more T follicular helper (Tfh) cells; and reintroducing the one or more activated CD4⁺ T cells into the subject.

### BRIEF DESCRIPTION OF THE DRAWINGS

For a more complete understanding of the features and advantages of the present invention, reference is now made to the detailed description of the invention along with the accompanying figures and in which:

FIGS. 1A-1K are images of plots illustrating the blood CXCR5 (X5)+CD4+ T cells induce naïve B cells to become Ig-producing plasmablasts.

FIGS. 2A-2N are images of plots illustrating the blood X5+ CD4+ T cells depends on IL-21, IL-10 and ICOS for B cell help.

FIGS. 3A-3F are images of plots illustrating Blood X5+ CD4+ T cells are composed of subsets.

FIGS. 4A-4G are images of plots illustrating X5+ Th2 and X5+ Th17 cells efficiently help naïve B cells.

FIGS. 5A-5I are images of plots illustrating blood X5+ Th subsets are altered in JDM.

FIGS. 6A-6F are images of plots illustrating skewing in blood X5+ CD4+ T cell subsets correlates with B cell alteration.

FIG. 7 is an image of plots illustrating the naïve CD4+ T cells stimulated with the combination of IL-6/TGF-b/IL-12 expressed less IFN-g than those stimulated with IL-12 alone or the combination of IL-12/TGF-b.

FIG. 8 is an image of the combination of IL-6/TGF-b/IL-12 yielded more CD4+ T cells expressing IL-21 but not IFN-g, a cytokine expression profile of human tonsillar Tfh cells.

FIG. 9 is an image of a plot illustrating CD4+ T cells stimulated with the combination of IL-6/TGF-b/IL-12 expressed higher levels of CXCR5 than those stimulated with IL-12 alone.

FIG. 10 is a plot of CD4+ T cells stimulated with the combination of IL-6/TGF-b/IL-12 expressed higher levels of Bcl-6 and lower levels of Blimp-1 than those stimulated with IL-12 alone or the combination of IL-12/TGF-b.

FIG. 11 is a graph of CD4+ T cells stimulated with the combination of IL-6/TGF-b/IL-12 induced B cells to produce higher levels of IgG than those stimulated with IL-12 alone or the combination of IL-12/TGF-b.

FIG. 12 is a graph of STAT3 siRNA transfection resulted in the strong inhibition of the development of IFN-g-IL-21+ cells, while promoting IFN-g+IL-21− Th1 cells.

FIGS. 13A-13I are images of plots illustrating blood X5+ Th subsets are altered in juvenile dermatomyositis (JDM) (blood CXCR5⁺ CD4⁺ T cell subsets are skewed towards Th2 and Th17 cells): FIG. 13A shows the percentage of CXCR5+ cells within CD4+ T cells in samples from JDM patients (n=52), PSOA patients (n=31), and age-matched healthy controls (n=43), FIG. 13B shows the percentage of each Th subset within blood CXCR5⁺ CD4⁺ T cells. One way ANOVA test, ** p<0.01, *** p<0.001, FIG. 13C shows the ratio of CXCR5⁺ (Th2+ Th17)/Th1 cells. One way ANOVA test, FIG. 13D shows the frequency of CXCR5⁺ Th1 cells and ratio of (Th2+Th17)/Th1 in JDM patients with different disease activities. One way ANOVA test, FIG. 13E shows the frequency of CXCR5⁺ Th1 cells and ratio of (Th2+Th17)/Th1 in active JDM patients receiving different treatments, FIG. 13F shows the absolute cell numbers in blood were calculated based on the complete blood cell count, lymphocyte frequency within white blood cells, and the frequency of CXCR5⁺ Th subsets within the lymphocyte population. Student's t-test, FIG. 13G shows the percentage of each Th subset within blood CXCR5− CD4+ T cells. One way ANOVA test, ** p<0.01, *** p<0.001, FIG. 13H shows the frequency of CXCR5+ Th1 cells and ratio of (Th2+Th17)/Th1 in PSOA patients receiving different treatments, and FIG. 13I shows the serum IgG, A, M, E levels in the three groups.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

The term “antigen” as used herein refers to a molecule that can initiate a humoral and/or cellular immune response in a recipient of the antigen. Antigen may be used in two different contexts with the present invention: as a target for the antibody or other antigen recognition domain or as an agent that causes an immune response, e.g., a T cell or a B cell response. When an antigen is presented on a Major Histocompatibility Complex (MHC) protein or proteins, the peptide is often about 8 to about 25 amino acids. Antigens include any type of biologic molecule, including, for example, simple intermediary metabolites, sugars, lipids and hormones as well as macromolecules such as complex carbohydrates, phospholipids, nucleic acids and proteins, and generally, these antigens are three dimensional targets for the binding of an antibody. Common categories of antigens include, but are not limited to, viral antigens, bacterial antigens, fungal antigens, protozoal and other parasitic antigens, tumor antigens, antigens involved in autoimmune disease, allergy and graft rejection, and other miscellaneous antigens.

The present invention also includes combinations with numerous active agents, e.g., antibiotics, anti-infective agents, antiviral agents, anti-tumoral agents, antipyretics, analgesics, anti-inflammatory agents, therapeutic agents for osteoporosis, enzymes, cytokines, anticoagulants, polysaccharides, collagen, cells, and combinations of two or more of the foregoing active agents. Examples of antibiotics for delivery using the present invention include, without limitation, tetracycline, aminoglycosides, penicillins, cephalosporins, sulfonamide drugs, chloramphenicol sodium succinate, erythromycin, vancomycin, lincomycin, clindamycin, nystatin, amphotericin B, amantidine, idoxuridine, p-amino salicyclic acid, isoniazid, rifampin, antinomycin D, mithramycin, daunomycin, adriamycin, bleomycin, vinblastine, vincristine, procarbazine, imidazole carboxamide, and the like.

Examples of cytokines include, without limitation, interleukins, transforming growth factors (TGFs), fibroblast growth factors (FGFs), platelet derived growth factors (PDGFs), epidermal growth factors (EGFs), connective tissue activated peptides (CTAPs), osteogenic factors, and biologically active analogs, fragments, and derivatives of such growth factors. Cytokines may be B/T-cell differentiation factors, B/T-cell growth factors, mitogenic cytokines, chemotactic cytokines, colony stimulating factors, angiogenesis factors, IFN-α, IFN-β, IFN-γ, IL1, IL2, IL3, IL4, IL5, IL6, IL7, IL8, IL9, IL10, IL11, IL12, IL13, IL14, ILLS, IL16, IL17, IL18, etc., leptin, myostatin, macrophage stimulating protein, platelet-derived growth factor, TNF-α, TNF-β, NGF, CD40L, CD137L/4-1BBL, human lymphotoxin-β, G-CSF, M-CSF, GM-CSF, PDGF, IL-1α, IL1-β, IP-10, PF4, GRO, 9E3, erythropoietin, endostatin, angiostatin, VEGF or any fragments or combinations thereof. Other cytokines include members of the transforming growth factor (TGF) supergene family include the beta transforming growth factors (for example TGF-β1, TGF-β2, TGF-β3); bone morphogenetic proteins (for example, BMP-1, BMP-2, BMP-3, BMP-4, BMP-5, BMP-6, BMP-7, BMP-8, BMP-9); heparin-binding growth factors (for example, fibroblast growth factor (FGF), epidermal growth factor (EGF), platelet-derived growth factor (PDGF), insulin-like growth factor (IGF)); Inhibins (for example, Inhibin A, Inhibin B); growth differentiating factors (for example, GDF-1); and Activins (for example, Activin A, Activin B, Activin AB).

Examples of anticoagulants for delivery using the present invention include, without limitation, include warfarin, heparin, Hirudin, and the like. Examples of factors acting on the immune system include for delivery using the present invention include, without limitation, factors which control inflammation and malignant neoplasms and factors which attack infective microorganisms, such as chemotactic peptides and bradykinins.

Examples of viral antigens include, but are not limited to, e.g., retroviral antigens such as retroviral antigens from the human immunodeficiency virus (HIV) antigens such as gene products of the gag, pol, and env genes, the Nef protein, reverse transcriptase, and other HIV components; hepatitis viral antigens such as the S, M, and L proteins of hepatitis B virus, the pre-S antigen of hepatitis B virus, and other hepatitis, e.g., hepatitis A, B, and C, viral components such as hepatitis C viral RNA; influenza viral antigens such as hemagglutinin and neuraminidase and other influenza viral components; measles viral antigens such as the measles virus fusion protein and other measles virus components; rubella viral antigens such as proteins E1 and E2 and other rubella virus components; rotaviral antigens such as VP7sc and other rotaviral components; cytomegaloviral antigens such as envelope glycoprotein B and other cytomegaloviral antigen components; respiratory syncytial viral antigens such as the RSV fusion protein, the M2 protein and other respiratory syncytial viral antigen components; herpes simplex viral antigens such as immediate early proteins, glycoprotein D, and other herpes simplex viral antigen components; varicella zoster viral antigens such as gpI, gpII, and other varicella zoster viral antigen components; Japanese encephalitis viral antigens such as proteins E, M-E, M-E-NS1, NS1, NS1-NS2A, 80% E, and other Japanese encephalitis viral antigen components; rabies viral antigens such as rabies glycoprotein, rabies nucleoprotein and other rabies viral antigen components. See Fundamental Virology, Second Edition, eds. Fields, B. N. and Knipe, D. M. (Raven Press, New York, 1991) for additional examples of viral antigens.

Antigens involved in autoimmune diseases, allergy, and graft rejection can be used in the compositions and methods of the invention. For example, an antigen involved in any one or more of the following autoimmune diseases or disorders can be used in the present invention: diabetes, diabetes mellitus, arthritis (including rheumatoid arthritis, juvenile rheumatoid arthritis, osteoarthritis, psoriatic arthritis), multiple sclerosis, myasthenia gravis, systemic lupus erythematosis, autoimmune thyroiditis, dermatitis (including atopic dermatitis and eczematous dermatitis), psoriasis, Sjogren's Syndrome, including keratoconjunctivitis sicca secondary to Sjogren's Syndrome, alopecia greata, allergic responses due to arthropod bite reactions, Crohn's disease, aphthous ulcer, iritis, conjunctivitis, keratoconjunctivitis, ulcerative colitis, asthma, allergic asthma, cutaneous lupus erythematosus, scleroderma, vaginitis, proctitis, drug eruptions, leprosy reversal reactions, erythema nodosum leprosum, autoimmune uveitis, allergic encephalomyelitis, acute necrotizing hemorrhagic encephalopathy, idiopathic bilateral progressive sensorineural hearing loss, aplastic anemia, pure red cell anemia, idiopathic thrombocytopenia, polychondritis, Wegener's granulomatosis, chronic active hepatitis, Stevens-Johnson syndrome, idiopathic sprue, lichen planus, Crohn's disease, Graves ophthalmopathy, sarcoidosis, primary biliary cirrhosis, uveitis posterior, and interstitial lung fibrosis. Examples of antigens involved in autoimmune disease include glutamic acid decarboxylase 65 (GAD 65), native DNA, myelin basic protein, myelin proteolipid protein, acetylcholine receptor components, thyroglobulin, and the thyroid stimulating hormone (TSH) receptor. Examples of antigens involved in allergy include pollen antigens such as Japanese cedar pollen antigens, ragweed pollen antigens, rye grass pollen antigens, animal derived antigens such as dust mite antigens and feline antigens, histocompatiblity antigens, and penicillin and other therapeutic drugs. Examples of antigens involved in graft rejection include antigenic components of the graft to be transplanted into the graft recipient such as heart, lung, liver, pancreas, kidney, and neural graft components. The antigen may be an altered peptide ligand useful in treating an autoimmune disease.

As used herein, the term “epitope(s)” refer to a peptide or protein antigen that includes a primary, secondary or tertiary structure similar to an epitope located within any of a number of pathogen polypeptides encoded by the pathogen DNA or RNA. The level of similarity will generally be to such a degree that monoclonal or polyclonal antibodies directed against such polypeptides will also bind to, react with, or otherwise recognize, the peptide or protein antigen. Various immunoassay methods may be employed in conjunction with such antibodies, such as, for example, Western blotting, ELISA, RIA, and the like, all of which are known to those of skill in the art. The identification of pathogen epitopes, and/or their functional equivalents, suitable for use in vaccines is part of the present invention. Once isolated and identified, one may readily obtain functional equivalents. For example, one may employ the methods of Hopp, as taught in U.S. Pat. No. 4,554,101, incorporated herein by reference, which teaches the identification and preparation of epitopes from amino acid sequences on the basis of hydrophilicity. The methods described in several other papers, and software programs based thereon, can also be used to identify epitopic core sequences (see, for example, Jameson and Wolf, 1988; Wolf et al., 1988; U.S. Pat. No. 4,554,101). The amino acid sequence of these “epitopic core sequences” may then be readily incorporated into peptides, either through the application of peptide synthesis or recombinant technology.

The preparation of vaccine compositions that includes the active ingredient, may be prepared as injectables, either as liquid solutions or suspensions; solid forms suitable for solution in, or suspension in, liquid prior to infection can also be prepared. The preparation may be emulsified, encapsulated in liposomes. The active immunogenic ingredients are often mixed with carriers which are pharmaceutically acceptable and compatible with the active ingredient.

The term “pharmaceutically acceptable carrier” refers to a carrier that does not cause an allergic reaction or other untoward effect in subjects to whom it is administered. Suitable pharmaceutically acceptable carriers include, for example, one or more of water, saline, phosphate buffered saline, dextrose, glycerol, ethanol, or the like and combinations thereof. In addition, if desired, the vaccine can contain minor amounts of auxiliary substances such as wetting or emulsifying agents, pH buffering agents, and/or adjuvants which enhance the effectiveness of the vaccine. Examples of adjuvants that may be effective include but are not limited to: aluminum hydroxide, N-acetyl-muramyl-L-threonyl-D-isoglutamine (thr-MDP), N-acetyl-nor-muramyl-L-alanyl-D-isoglutamine, MTP-PE and RIBI, which contains three components extracted from bacteria, monophosphoryl lipid A, trehalose dimycolate and cell wall skeleton (MPL+TDM+CWS) in a 2% squalene/Tween 80 emulsion. Other examples of adjuvants include DDA (dimethyldioctadecylammonium bromide), Freund's complete and incomplete adjuvants and QuilA. In addition, immune modulating substances such as lymphokines (e.g., IFN-γ, IL-2 and IL-12) or synthetic IFN-γ inducers such as poly I:C can be used in combination with adjuvants described herein.

T follicular helper (Tfh) cells represent a subset of CD4+ T cells, which express chemokine (C-X-C motif) receptor 5 (CXCR5), and are specialized for B cell help in germinal centers (GCs). While a fraction of human blood memory CD4+ T cells expresses CXCR5, their relationship to Tfh cells is not well-established. Here we show that human blood CXCR5+ CD4+T cells largely share the functional properties with Tfh cells, and represent their circulating memory compartment. Blood CXCR5+ CD4+ T cells comprise three subsets, Th1, Th2 and Th17 cells, which can be distinguished based on chemokine receptor expression. Th2 and Th17 cells within CXCR5+, but not CXCR5−, compartment efficiently induce naïve B cells to produce immunoglobulins via Interleukin (IL)-21. CXCR5+ Th17 cells were potent at inducing IgA response. In contrast, Th1 cells, from both CXCR5+ and CXCR5− compartments, lack the capacity to help B cells. Patients with juvenile dermatomyositis, a systemic autoimmune disease, displayed a profound skewing of blood CXCR5+ CD4+ T cell subsets towards Th2 and Th17. Significantly, the skewing of subsets correlated with disease activity and frequency of blood plasmablasts. Collectively, these observations suggest that an altered balance of Tfh subsets contributes to human autoimmunity.

Antibody response is largely dependent on the help provided by CD4+ T cells (Clark and Ledbetter, 1994). Early in vitro studies using clones and recombinant cytokines have concluded that multiple CD4+ T cell subsets contribute to the regulation of antibody response (Mosmann and Coffman, 1989). IFN-β secreted by T helper (Th)1 cells induces mouse B cell class-switching towards IgG2a, while IL-4 and IL-13 secreted by Th2 cells promote class-switching towards IgG1 and IgE. CD4+ T cells are fundamental for the generation of germinal centers (GCs), a discrete structure in secondary lymphoid organs where selection of high-affinity B cells and development of B cell memory occur (Allen et al., 2007; MacLennan, 1994). CD4+ T cells present in B cell follicles, named T follicular helper cells (Tfh), have been recently established as a Th subset specialized for providing help to B cells at GCs (Fazilleau et al., 2009; King et al., 2008; Vinuesa et al., 2005b). Tfh cells express the chemokine (C-X-C motif) receptor 5 (CXCR5) (Breitfeld et al., 2000; Kim et al., 2001b; Schaerli et al., 2000), which allows their migration into follicles in response to the specific ligand, CXCL13 secreted by follicular dendritic cells (Ansel et al., 1999). Tfh cells secrete IL-4, IL-10 and IL-21, cytokines that efficiently promote growth, differentiation, and class-switching of B cells (Ettinger et al., 2005; Good et al., 2006; King and Mohrs, 2009; Moore et al., 1993; Pene et al., 2004; Zaretsky et al., 2009). Tfh cells also express multiple surface molecules essential for the helper functions, including CD40-ligand (CD40L), and inducible co-stimulator (ICOS) (King et al., 2008). Tfh cells express high levels of B cell lymphoma 6 (Bcl-6) (Chtanova et al., 2004; Rasheed et al., 2006), a transcription factor essential for the development of GC B cells (Dent et al., 1997; Ye et al., 1997). Recent mouse studies showed that Bcl-6 is necessary and sufficient for the development of Tfh cells in vivo (Johnston et al., 2009; Nurieva et al., 2009; Yu et al., 2009), and thus represents a master transcription factor controlling Tfh generation. In contrast, B lymphocyte-induced maturation protein 1 (Blimp-1), a transcription factor that represses the function of Bcl-6, inhibits the generation of Tfh cells (Johnston et al., 2009). Thus, Tfh generation is controlled by the balance of these two transcription factors. The developmental pathway of Tfh cells differs from that of Th1, Th2, and Th17 cells (Nurieva et al., 2008). There is evidence that mouse Tfh cells are heterogeneous, and encompass distinct subsets secreting cytokines characteristic of Th1, Th2, and Th17 cells (Bauquet et al., 2009; Fazilleau et al., 2009; King and Mohrs, 2009; Reinhardt et al., 2009; Zaretsky et al., 2009). Furthermore, mouse Th2 (Zaretsky et al., 2009) and T regs (Tsuji et al., 2009) were shown to be convertible into Tfh cells in vivo.

Previous studies on Tfh cells from human secondary organs such as tonsils or spleen have concluded that Tfh cells display distinct phenotype and gene profiles from other conventional Th subsets (Chtanova et al., 2004; Kim et al., 2004; Rasheed et al., 2006). This might be at least due to the over-representation of Bcl-6, which represses many genes including transcription factors expressed by canonical Th subsets (Nurieva et al., 2009; Yu et al., 2009).

Several mouse studies show that over-representation of IL-21-producing Tfh cells is associated with the development of systemic autoimmunity (Linterman et al., 2009; Subramanian et al., 2006; Vinuesa et al., 2005a). A feature of autoimmune disease is the presence of autoantibodies reacting to multiple self antigens (Martin and Chan, 2006). Patients with autoimmune diseases such as lupus or rheumatoid arthritis display autoreactive naïve B cells at significantly higher frequencies than healthy subjects (Samuels et al., 2005; Yurasov et al., 2005), due to the alteration in checkpoints of B cell tolerance (Meffre and Wardemann, 2008). The emergence of high-affinity, somatically mutated autoantibodies (Mietzner et al., 2008; Shlomchik et al., 1987) in such patients suggest the involvement of Tfh cells in the generation of autoantibody producing cells.

A systematic approach would be appropriate to determine the role of Tfh cells in the human autoimmunity, as analysis of lymph nodes and/or tonsil samples sporadically obtained from autoimmune disease patients might be misleading. It is difficult, however, to routinely and longitudinally obtain secondary lymphoid organ samples from patients. Therefore, there is a strong need to establish surrogate strategies to assess the quality of Tfh responses in humans. Regarding this issue, detailed analysis of blood CD4⁺ T cells expressing CXCR5 (Forster et al., 1994) facilitates studies on Tfh responses in humans. Several observations suggest the relationship between CXCR5⁺ CD4⁺ T cells and Tfh cells developed via GC reactions. For example, the levels of circulating CXCR5⁺ CD4⁺ T cells are significantly decreased in humans who display severely impaired GC formation through deficiency of functional CD40-ligand, or ICOS (Bossaller et al., 2006). On the contrary, circulating CXCR5⁺ CD4⁺ T cells expressing ICOS are present at a higher frequency in patients with systemic lupus erythematosus (Simpson et al., 2010).

### Example 1

### Human Blood CXCR5⁺ CD4⁺ T Cells Represent a Circulating Pool of Memory Tfh Cells

It is demonstrated herein that human blood CXCR5⁺ CD4⁺ T cells represent a circulating pool of memory Tfh cells. Significantly, blood CXCR5⁺ CD4⁺ T cells can be distinguished into Th1, Th2, and Th17 populations according to chemokine receptor expression and by the type of cytokine secretion patterns. These subsets differentially regulate the differentiation and class-switching of naïve B cells. Finally, we show an alteration of blood CXCR5⁺ CD4⁺ T cell subsets in an autoimmune disease, juvenile dermatomyositis (JDM).

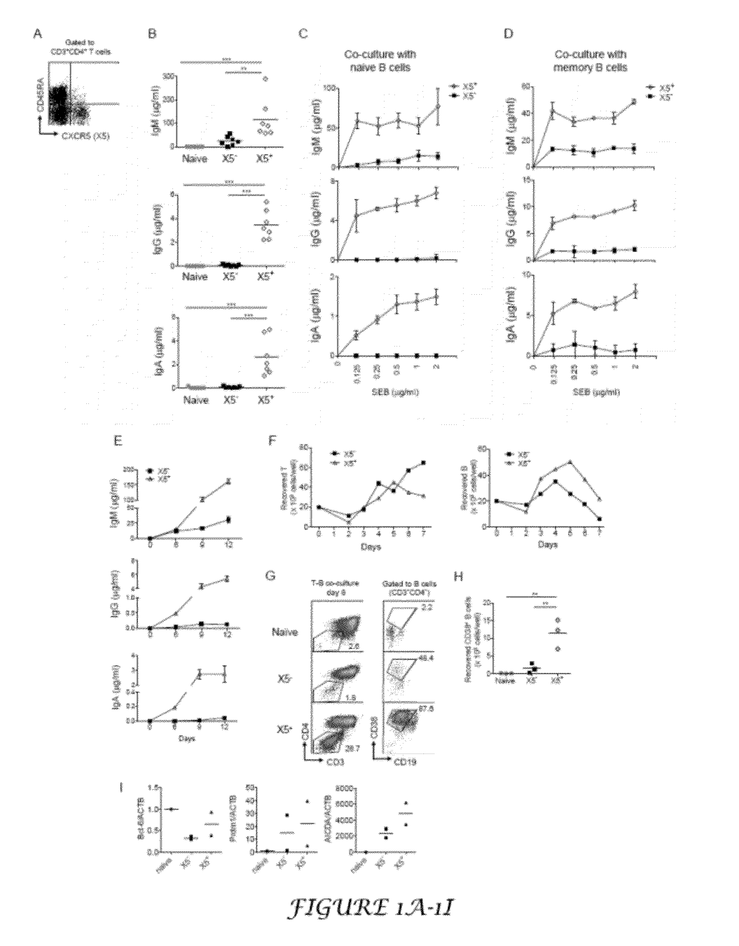

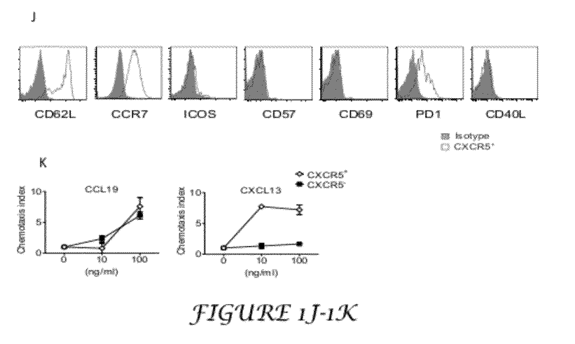

FIGS. 1A-1I are images of plots illustrating the blood CXCR5⁺ (hereunder called X5) CD4⁺ T cells induce naïve B cells to become Ig-producing plasmablasts. Human blood CXCR5+ CD4+ T cells induce the differentiation of naïve B cells towards plasmablasts. X5 expression by blood CD4+ T cells. FIG. 1A is an image of a plot showing, Ig secretion. Blood naive, X5⁻, and X5⁺ CD4⁺ T cells were cultured with autologous naïve B cells in the presence of SEB. The levels of produced Igs were measured at day 12. One way ANOVA test. n=7. ** p<0.01, *** p<0.001. Titration of SEB. X5⁻ or X5⁺ CD4⁺ T cells were cultured with naïve B cells in the presence of titrated doses of SEB, and the levels of produced Igs were measured at day 12. FIG. 1A is an image of a plot showing Ig secretion by memory B cells. X5⁻ or X5⁺ CD4⁺ T cells were cultured with memory B cells in the presence of titrated doses of SEB, and the levels of produced Igs were measured at day 6. FIG. 1B is an image of a plot showing the kinetics of Ig production. The levels of produced Igs were measured at indicated days in the cultures of X5⁻ or X5⁺ CD4⁺ T cells with naïve B cells. FIG. 1C is an image of a plot showing the recovery of viable T and B cells. Co-cultured cells were harvested at the indicated time points, and number of viable cells per well were determined with trypan-blue staining Cells were stained with anti-CD3 and CD4 mAbs, and CD4⁺ T and B cell populations were analyzed by flow cytometry. FIG. 1D is an image of a plot showing the CD38⁺ plasmablast population in the co-culture of CD4⁺ T cells and naive B cells was analyzed at day 8. FIG. 1E is an image of a plot showing the absolute number of plasmablasts in the co-cultures. One way ANOVA test and n=3. FIG. 1D is an image of a plot showing the expression levels of Bcl-6, Prdm1, and AICDA mRNA by naïve B cells cultured for 6 d with either X5⁻ or X5⁺ CD4⁺ T cells were analyzed by real-time RT-PCR. In healthy adult blood, X5 was expressed by 8.3±1.8% of total CD4⁺ T cells, and 18.9±3.6% of memory CD45RA⁻ CD4⁺ T cells (mean±s.d., n=10)(as seen in FIG. 1A). Consistent with previous observations (Breitfeld et al., 2000; Kim et al., 2001b; Schaerli et al., 2000), blood X5⁺ CD4⁺ T cells expressed CCR7 and CD62L, but few expressed activation molecules expressed by Tfh cells, such as ICOS and CD69, suggesting a resting state. FIG. 1J is a plot of the blood CXCR5 (CXCR5)+CD4+ T cells do not express activation markers. The expression of each molecule was analyzed by FACS. Gated to CD3+CD4+CXCR5+ T cells. FIG. 1K is a plot of the blood CXCR5+ CD4+ T cells that migrate in response to CCL19 and CXCL13. Migration of CD4+ T cells was assayed by using Boyden chamber (Neuro Probe). CXCL13 or CCL19 (10 and 100 ng/ml; both from R&D Systems) was added to the lower chamber (27 μl), while CD4+ T cells (8.5×10⁵ cells/ml) were added to the upper chamber (50 μl) separated with polycarbonate filter with 5 μm pores. The chamber was incubated 2 h at 37° C., and the filters were stained with Diff-Quik solutions (Dade Behring) after the upper side was cleaned. Migrating cells were counted four fields at ×200 magnification per well. The data were expressed as chemotaxis index calculated with this formula: chemotaxis index=the number of migrating cells exposed to the chemokines/the number of cells in background. n=3, Mean±s.d. Both CXCR5 and CCR7 are functional, as blood X5⁺ CD4⁺ T cells migrated in response to the ligands, CXCL13 and CCL19, respectively.

To determine their capacity to help B cells, memory X5⁺ cells (CD45RA⁻X5⁺) were sorted, and cultured with autologous naïve B cells (IgD⁺CD27⁻CD19⁺ cells). Naïve CD4⁺ T cells (CD45RA^{+X}5⁻) and memory X5⁻ cells (CD45RA⁻X5⁻) were also sorted as controls. To mimic the antigen-specific interaction between T and B cells, staphylococcal enterotoxin B (SEB), a superantigen, was added to the cultures. Naïve CD4⁺ T cells did not induce naïve B cells to produce immunoglobulins (Igs), as seen in FIG. 1B. Memory X5⁻ CD4⁺ T cells induced naïve B cells to produce only low amounts of IgM (25±7 μg/ml. Mean±s.e.m. n=7), but no IgG or IgA. In contrast, X5⁺ CD4⁺ T cells were potent at inducing naive B cells to produce Igs, including IgM (118±32 μg/ml; mean±s.e.m. n=7), IgG (3.5±0.5 μg/ml), and IgA (2.7±0.6 μg/ml). The Ig production was totally dependent on cognate interactions between T and B cells, as naïve B cells co-cultured with X5⁺ CD4⁺ T cells did not produce Igs in the absence of SEB, as seen in FIG. 1C. X5⁺ CD4⁺ T cells were also more efficient in inducing memory B cells to produce Igs when compared to X5⁻ CD4⁺ T cells, as seen in FIG. 1D.

Kinetics studies revealed that X5⁺ CD4⁺ T cells induced naïve B cells to produce IgG and IgA as early as day 6 of culture, while X5⁻ CD4⁺ T cells did not induce either IgG or IgA secretion even at day 12, as seen in FIG. 1E. The number of viable T cells was similar between X5⁻ and X5⁺ CD4⁺ T cells in the cultures with naïve B cells, as seen in FIG. 1F, left, indicating that the inability of X5⁻ CD4⁺ T cells to induce naïve B cells to produce IgG or IgA was not due to their poor survival in culture. In contrast, the number of viable B cells was constantly higher when co-cultured with X5⁺ CD4⁺ T cells than with X5⁻CD4⁺ T cells, as seen in FIG. 1F, right. Furthermore, culturing naïve B cells with X5⁺ CD4⁺ T cells yielded higher numbers of CD38⁺ CD19^{dim} plasmablasts than with X5⁻CD4+ T cells, as seen in FIGS. 1G and 1H. Both X5⁺ and X5⁻ CD4⁺ T cells induced naïve B cells to express activation-induced cytidine deaminase (AID, encoded by AICDA gene), a factor required for class-switching, and Blimp-1 (encoded by Prdm1 gene), a transcription factor critical for the differentiation of plasma cells, but not Bcl6, as seen in FIG. 1I. These observations show that blood X5⁺ CD4⁺ T cells are more efficient than X5⁻ CD4⁺ T cells at inducing naive B cells to differentiate into Ig-secreting plasmablasts and to promote class switching.

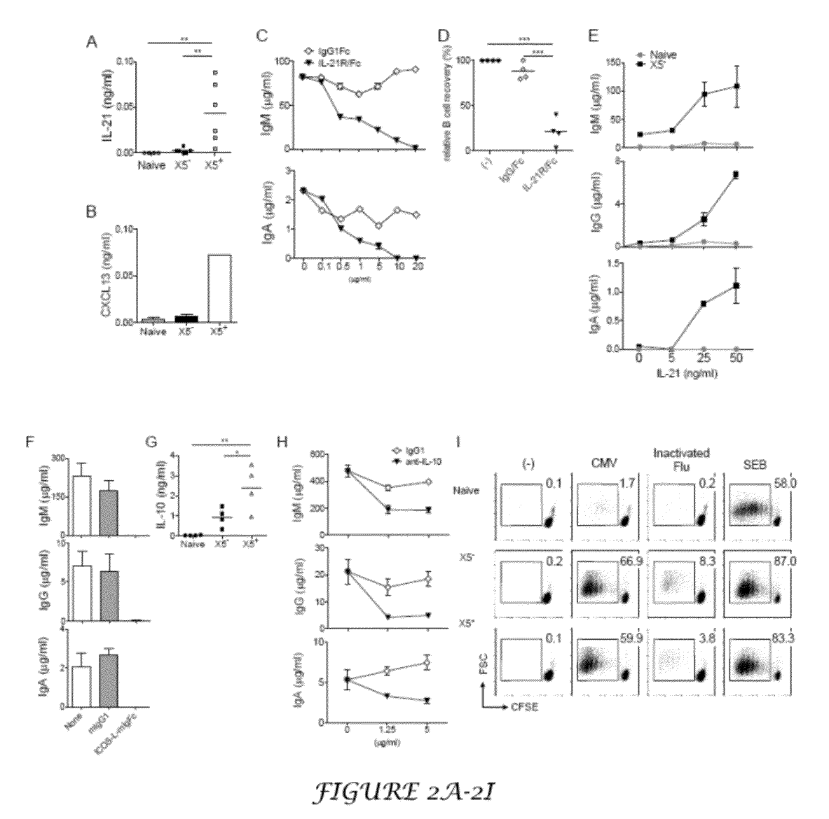

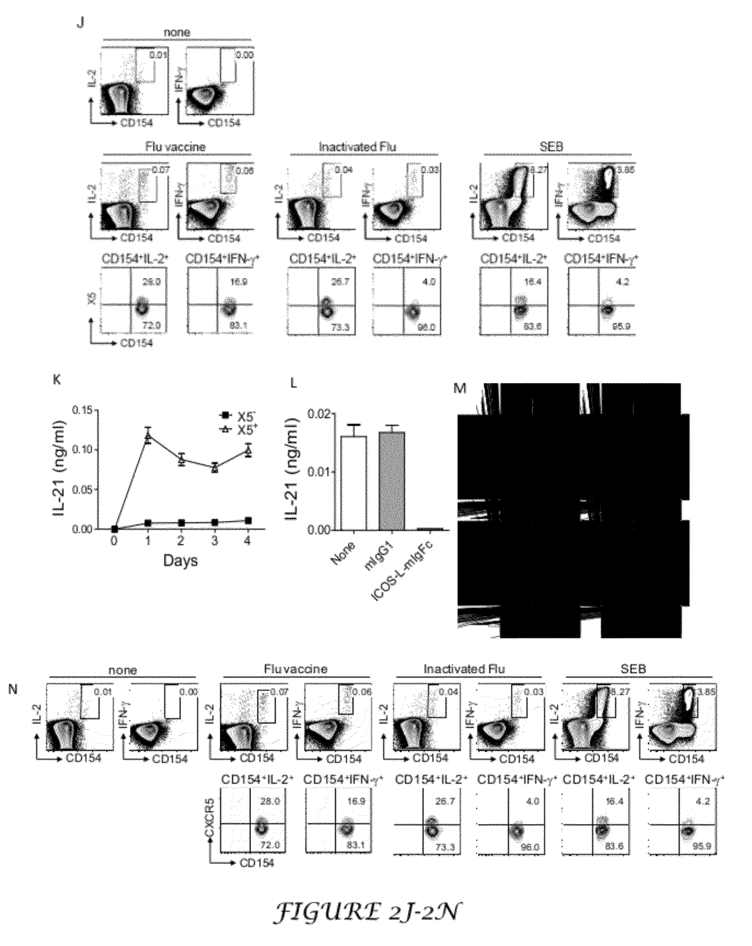

FIGS. 2A-2J are images of graphs illustrating blood X5+ CD4+ T cells depends on IL-21, IL-10 and ICOS for B cell help. FIG. 2A is an image of a graph illustrating IL-21 levels in supernatants of blood CD4+ T cells cultured for 48 hours with naïve B cells. One way ANOVA test, n=6 and p<0.01. FIG. 2B is an image of a graph illustrating CXCL13 levels in supernatants of blood CD4+ T cells cultured for 48 hours with naïve B cells. FIG. 2C is an image of a graph illustrating IL-21 blocking Titrated amounts of IL-21R/Fc or IgG1Fc were added to the co-culture of X5⁺CD4⁺ T cells and naïve B cells. Produced Igs were measured at day 12. IgG is not shown due to the cross-reactivity of Fc portion of the chimeric proteins to anti-human IgG mAb.

FIG. 2D is an image of a graph illustrating the recovery of B cells. The number of viable B cells at day 12 of co-cultured was analyzed. One way ANOVA test, n=4 and p<0.001. FIG. 2E is an image of a graph illustrating IL-21 supplementation. Titrated amounts of IL-21 were added to the co-culture of naïve or X5− CD4+ T cells with naïve B cells. Ig levels at day 12. FIG. 2F is an image of a graph illustrating ICOS blocking ICOSL-mIgFc or mIgG1 was added to the co-culture of X5⁺CD4⁺ T cells and naïve B cells. Ig levels at day 12. FIG. 2G is an image of a graph illustrating IL-10 levels in supernatants of blood CD4+ T cells cultured for 48 hours with naïve B cells. One way ANOVA test, n=4 * p<0.05, ** p<0.01. FIG. 2H is an image of a graph illustrating IL-10 blocking. Indicated amounts of IL-10 neutralizing mAb or IgG1 were added to the co-culture of X5⁺CD4⁺ T cells and naïve B cells. Ig levels at day 12. FIG. 2I is an image of a graph illustrating microbe-specific memory cells. CFSE-labeled blood naïve, X5+, and X5− CD4+ T cells were cultured with autologous monocytes incubated with inactivated influenza virus or CMV. Cell proliferation was analyzed at day 5 by flow cytometry. FIG. 2J is an image of a graph illustrating expression of CD154 and cytokines PBMCs were stimulated with a Flu vaccine or inactivated influenza virus for 6 hours in the presence of Brefeldin A and monensin, and the intracytoplasmic expression of CD154, IL-2, and IFN-γ in X5+ or X5− CD4+ T cells was analyzed. Expression of X5 was analyzed within the CD4+ T cell population expression CD154 and cytokines (bottom).

Similar to tonsillar Tfh cells, X5⁺ CD4⁺ T cells secreted IL-21 upon contact with naïve B cells, while X5⁻ CD4⁺ T cells barely secreted IL-21, as seen in FIG. 2A. While X5⁺ CD4⁺ T cells secrete IL-21 within 24 hours after interaction with naïve B cells, very low, if any, levels of IL-21 were secreted by X5⁻ CD4⁺ T cells up to 96 hours. Notably, X5⁺ CD4⁺ T cells also secreted higher levels of CXCL13 than X5⁻ CD4⁺ T cells, as seen in FIG. 2B, a chemokine secreted by tonsillar Tfh cells (Kim et al., 2004; Rasheed et al., 2006).

IL-21 secreted by tonsillar Tfh cells plays a central role in the expansion and plasma cell differentiation of co-cultured B cells (Bryant et al., 2007). To analyze whether blood X5⁺ CD4⁺ T cells share the same mechanism, IL-21 was blocked with an IL-21R-Fc chimeric protein during the co-cultures. This resulted in a dose-dependent inhibition of Ig secretion, as seen in FIG. 2C, and B cell recovery, as seen in FIG. 2D. Conversely, addition of IL-21 to co-cultures of X5⁻ CD4⁺ T cells and naïve B cells resulted in the enhancement of IgM secretion as well as the induction of IgG and IgA secretion, as seen in FIG. 2E. Naïve CD4⁺ T cells did not induce naïve B cells to produce Igs even in the presence of IL-21, showing the fundamental difference in functional properties between naïve and memory CD4⁺ T cells. B cell help by blood X5⁺ CD4⁺ T cells was also dependent on ICOS, as addition of an ICOS-L-Fc chimeric protein resulted in the inhibition of both the IL-21 secretion and Ig secretion, as seen in FIG. 2F. IL-10 was also detected at higher levels in the co-cultures of X5⁺ CD4⁺ T cells and naïve B cells, as seen in FIG. 2G. Blocking of IL-10 with an IL-10 neutralizing antibody resulted in a partial inhibition of Ig secretion by naïve B cells co-cultured with X5⁺ CD4⁺ T cells, as seen in FIG. 2H.

Whether blood X5⁺ CD4⁺ T cells contain antigen-specific memory cells has been controversial (Breitfeld et al., 2000; Rivino et al., 2004; Schaerli et al., 2001). To determine whether blood X5⁺ CD4⁺ T cells contain memory cells specific for microbial antigens, isolated X5⁻ and X5⁺ CD4⁺ T cells were labeled with CFSE, and co-cultured with autologous monocytes that had been pulsed with influenza virus, or cytomegalovirus. Proliferation of CD4⁺ T cells were analyzed by flow cytometry at day 5. X5⁺ CD4⁺ T cells proliferated robustly in response to the stimulation with both viruses. To illustrate the cytokine expression by X5⁺ CD4⁺ T cells in response to the stimulation with influenza virus antigens, PBMCs were obtained from donors who did not receive influenza vaccines more than 1 year, and were incubated for 6 hours with either a seasonal influenza (Flu) vaccine (Fluzone) or a heat-inactivated influenza virus in the presence of Brefeldin A and monensin. Then cells were analyzed for the expression of cell surface CD3, CD4, and X5 and intracytoplasmic IL-2 and IFN-γ together with CD154, the expression of which permits the sensitive detection of antigen-specific CD4⁺ T cells (Chattopadhyay et al., 2005; Frentsch et al., 2005). While X5 can be expressed by activated CD4⁺ T cells, X5⁻ CD4⁺ T cells stimulated for 6 hours with SEB remained negative for X5 expression. As shown in FIG. 2J, Flu-specific CD4⁺ T cells were detected as CD154⁺ cells expressing IL-2 and/or IFN-γ in both stimulations. CD154⁺IL-2⁺ and CD154⁺IFN-γ⁺ cells contained a fraction of CD4⁺ T cells expressing X5, showing the presence of X5⁺ CD4⁺ T cells specific for Flu antigens capable of secreting IL-2 and/or IFN-γ. FIG. 2K is a plot of the kinetics of IL-21 secretion by blood CXCR5+ CD4+ T cells. Isolated CXCR5+ and CXCR5−CD4+ T cells were cultured with autologous naïve B cells in the presence of SEB. The secreted IL-21 levels were analyzed at the indicated time points. n=3, Mean±s.d. FIG. 2L is a graph of the blocking ICOS inhibits IL-21 secretion by CXCR5+ CD4+ T cells. Blood CXCR5+ CD4+ T cells were cultured with SEB-pulsed naïve B cells in the presence of either control mIgG1 or ICOS-L-mIgFc. The levels of IL-21 in cultures were measured at day 2. n=3, Mean±s.d. FIG. 2M is an image of CXCR5− CD4+ T cells do not upregulate CXCR5 expression during 6 h stimulation with SEB. Isolated CXCR5+ and CXCR5− CD4+ T cells were cultured with autologous monocyte pulsed with SEB (1 μg/ml) or none for 6 h. Expression of CXCR5 and intracytoplasmic CD154 was analyzed by flow cytometry. FIG. 2N is a plot of the expression of CD154 and cytokines. PBMCs were stimulated with Flu vaccine or inactivated influenza virus for 6 h in the presence of Brefeldin A and monensin, and the intracytoplasmic expression of CD154, IL-2, and IFN-β in CXCR5+ or CXCR5− CD4+ T cells was analyzed. Expression of CXCR5 was analyzed within the CD4+ T cell population expression CD154 and cytokines (bottom).

These observations show that X5⁺ CD4⁺ T cells provide help to naïve B cells via IL-21, IL-10, and ICOS, and thus share the functional properties of Tfh cells. Inasmuch as X5⁺ CD4⁺ T cells contain antigen-specific memory cells, we conclude that blood circulating X5⁺ CD4⁺ T cells represent circulating memory Tfh cells.

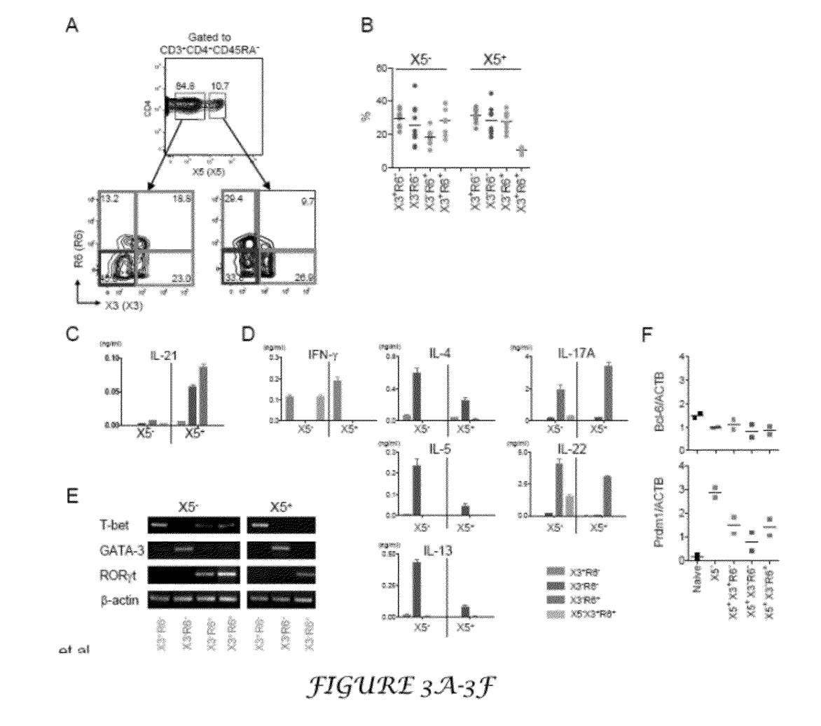

FIGS. 3A-3F are images of graphs illustrating that blood X5⁺ CD4⁺ T cells are composed of Three distinct Tfh cell subsets. FIG. 3A is an image of a graph illustrating subpopulations within X5⁻ and X5⁺ Th cells. CXCR3 (X3) and CCR6 (R6) expression was analyzed on CD3⁺CD4⁺CD45RA⁻X5⁻ or X5⁺ cell population by multicolor flow cytometry. FIG. 3B is an image of a graph illustrating frequency of subpopulations within blood X5⁻ and X5⁺ Th cells of 10 healthy adults. FIG. 3C is an image of a graph illustrating IL-21 secretion by Th subpopulations. Blood Th subpopulations (total 7 subpopulations) were co-cultured with naïve B cells and the secreted IL-21 was measured at 48 h. FIG. 3D is an image of a graph illustrating measurement of other cytokine levels secreted by blood Th subpopulations cultured with naïve B cells. FIG. 3E is an image of a graph illustrating expression of transcription factors. Expression of each transcriptional factor in the seven blood CD4⁺ T cell subpopulations was assessed by RT-PCR. FIG. 3F is an image of a graph illustrating expression of Bcl-6 and Prdm1 mRNA. Expression of Bcl-6 and Prdm-1 in X5⁻ CD4⁺ T cells and X5⁺ Th subsets was analyzed by real-time RT-PCR.

The expression of chemokine receptors, particularly in combination of several receptors (Kim et al., 2001a), has been instrumental for defining human CD4⁺ T cell subsets. The expression of CXCR3 (hereafter called X3) is preferentially maintained by cells committed to the Th1 pathway (Bonecchi et al., 1998; Rabin et al., 2003; Sallusto et al., 1998; Song et al., 2005), while CCR6 (hereafter called R6) is expressed by Th17 cells (Acosta-Rodriguez et al., 2007; Annunziato et al., 2007; Singh et al., 2008). Though blood X5⁺ CD4⁺ T cells were previously shown to co-express other chemokine receptors (Lim et al., 2008), the relationship between chemokine receptor expression and their function has not been established. As illustrated in FIG. 3A, differential expression of X3 and R6 defines three major subsets within blood X5⁺ CD4⁺ T cells: X3⁺R6⁻, X3⁻R6⁻, and X3⁻R6⁺ cells. In healthy adult blood, X3⁺R6⁻ T cells constituted 31±1% of X5⁺ CD4⁺ T cells, while X3⁻R6⁻ and X3⁻R6⁺ T cells represented 28±3% and 28±2%, respectively (as illustrated in FIG. 3B. Mean±s.e.m. n=10). X5⁻ CD4⁺ T cells contain four subpopulations including X3⁺R6⁺ cells. When compared to X5⁺ CD4⁺ T cells, X5⁻ CD4⁺ T cells contained less X3⁻R6⁺ T cells (18±1%, p<0.01, paired t-test) and more X3⁺R6⁺ T cells (X5⁻ 28±2% vs. X5⁺10±1%. p<0.001) (as illustrated in FIG. 3B).

To analyze the functional differences of blood memory CD4⁺ T cell populations, seven major subpopulations (four X5⁻ and three X5⁺ subpopulations) were isolated according to the expression of X5, X3 and R6. To analyze cytokine secretion patterns, each subpopulation was co-cultured for 2 days with SEB-pulsed naïve B cells as illustrated in FIG. 2A, all four subpopulations within the X5⁻ compartment secreted very little, if any, IL-21 upon interaction with naïve B cells (as illustrated in FIG. 3C). Within the X5⁺ CD4⁺ T cell compartment, only X3⁻R6⁻ and X3⁻R6⁺ cells produced IL-21, but X3⁺R6⁻ cells did not. Each cell population secreted different sets of cytokines X3⁺R6⁻ cells in X5⁺ as well as in X5⁻ CD4⁺ T cells secreted IFN-γ, but not Th2 or Th17 cytokines (as illustrated in FIG. 3D). Th2 cytokines, i.e., IL-4, IL-5, and IL-13, were exclusively secreted by X3⁻R6⁻ T cells in both X5⁺ and X5⁻ CD4⁺ T cells (as illustrated in FIG. 3D), while Th17 cytokines, IL-17A and IL-22, were produced by both X5⁺ X3⁻R6⁺ and X5⁻X3⁻R6⁺ T cells (as illustrated in FIG. 3D). The three subpopulations of X5⁺ CD4⁺ T cells expressed the transcription factors associated to the distinct Th lineages. Thus, X3⁺R6⁻ cells uniquely expressed T-bet, a transcription factor of Th1 cells, X3⁻R6⁻ cells expressed GATA3, a transcription factor of Th2 cells, while X3⁻R6⁺ cells expressed RORγT, a transcription factor of Th17 cells (as illustrated in FIG. 3E). The similar pattern of transcription factor expression was also observed within X5⁻ CD4⁺ T cell compartments. X3⁺R6⁺ cells in X5⁻ CD4⁺ T cells secreted primarily IFN-γ and IL-22, and expressed both T-bet and RORγT.

Next, we analyzed the expression level of Bcl-6 and Blimp-1 in blood Th subsets with real-time RT-PCR. Blood X5⁺ CD4⁺ T cells were shown to express much lower levels of Bcl-6 mRNA than tonsillar Tfh cells (Chtanova et al., 2004; Rasheed et al., 2006; Simpson et al., 2010). Indeed, the expression levels of Bcl-6 mRNA were similar among X5⁺ CD4⁺ T cell subsets and X5⁻ CD4⁺ T cells (as illustrated in FIG. 3F). However, the Blimp-1 mRNA expression was lower in X5⁺ Th subsets than X5⁻ CD4⁺ T cells as illustrated in FIG. 3F.

Collectively, both blood X5⁺ and X5⁻ CD4⁺ T cells include Th1, Th2, and Th17 cells. Neither X5⁺ nor X5⁻ Th1 cells secrete IL-21 by interacting with naïve B cells. X5⁺ Th2 and X5⁺ Th17 cells secrete IL-21, while X5⁻ Th2 and X5⁻ Th17 cells do not.

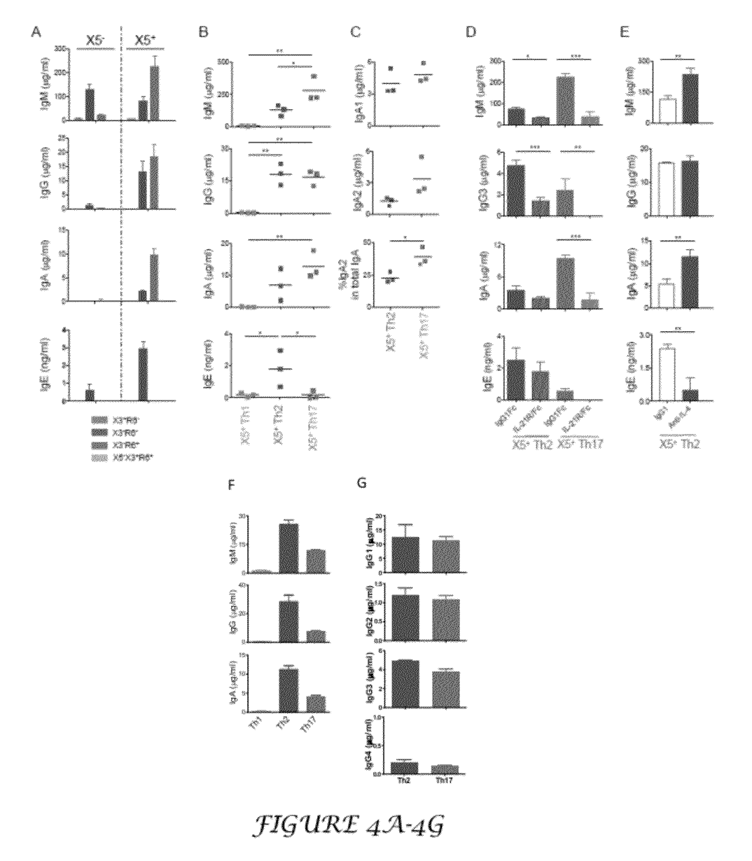

FIGS. 4A-4G are images of plots illustrating X5⁺ Th2 and X5⁺ Th17 cells help B cell differentiation. FIG. 4A is an image of a graph illustrating Ig secretion by naïve B cells co-cultured with blood CD4⁺ T cell subpopulations for 12 d. FIG. 4B is an image of a graph illustrating three independent studies of Ig secretion from naïve B cells co-cultured with blood X5+ CD4+ T cell subsets. One way ANOVA test, n=3. * p<0.05, ** p<0.01. FIG. 4C is an image of a graph illustrating IgA subclasses. The levels of produced IgA1 and IgA2 were analyzed. n=3. Student's t-test. * p<0.05. FIG. 4D is an image of a graph illustrating IL-21R/Fc chimera protein or IgG1Fc was added to the co-cultures of naïve B cells and X5+ Th2 (X3−R6−) or X5+ Th17 (X3−R6+) cells. For IgG, IgG3 levels were measured as the antibodies did not cross recognize IL-21R/Fc protein. Student's t-test. * p<0.05, ** p<0.01, *** p<0.001. FIG. 4E is an image of a graph illustrating IL-4 blocking antibody added to the culture of X5+ Th2 (X3−R6−) cells and naïve B cells. Student's t-test. ** p<0.01. FIG. 4F is an image of CXCR5⁺ Th1 (CXCR3⁺CCR6⁻) cells are unable to help memory B cells to produce Igs. Th subsets within blood CXCR5⁺ Th cells were co-cultured with SEB-pulsed memory B cells (IgD⁻ CD27⁺CD19⁺ B cells), and the produced Igs were measured at day 12. n=3-4, Mean±s.d. FIG. 4G is an image of production of IgG subclasses by naïve B cells co-cultured with CXCR5⁺ Th2 and CXCR5⁺ Th17 cells. Blood CXCR5⁺ Th2 or Th17 cells were co-cultured with SEB-pulsed naive B cells, and the produced IgG subclasses were measured at day 12. n=3, Mean±s.d.

The sorted CD4⁺ T cells were cultured with SEB-pulsed naïve B cells for 12 days, and secreted Ig levels were measured. Among Th1 (X3⁺R6⁻) cells, both X5⁺ and X5⁻ Th1 cells failed to induce naïve B cells to produce Igs. X5⁺ Th1 cells were also incapable of inducing memory B cells to produce Igs. Among Th2 (X3⁻R6⁻) cells, X5⁺ Th2 cells induced naïve B cells to produce large amounts of Igs, including IgM, IgG, IgA, and IgE. As illustrated in FIG. 4A, X5⁻ Th2 cells induced B cells to secrete only IgM and small amounts of IgE, but no IgG and IgA. Among Th17 (X3⁻R6⁺) cells, X5⁺ Th17 cells potently induced naïve B cells to produce IgM, and to undergo isotype switching towards IgG and IgA, but not IgE. However, X5⁻ Th17 (X3⁻R6⁺) cells completely lacked the capacity to help naïve B cells to produce any Igs as illustrated in FIGS. 4A and 4B.

While X5⁺ Th2 cells and X5⁺ Th17 cells induced naïve B cells to produce comparable levels of IgG (as illustrated in 4B) and IgG subclasses, X5⁺ Th17 cells induced B cells to produce IgA than did X5⁺ Th2 cells (as illustrated in FIG. 4B). In particular, X5⁺ Th17 cells yielded higher levels of IgA2 production than X5⁺ Th2 cells (as illustrated in FIG. 4C). Blocking IL-21 with IL-21R/Fc chimeric protein in the culture of naïve B cells with X5⁺ Th2 cells resulted in a significant decrease in IgM and IgG3 (the antibodies of which do not crossreact with IL-21R/Fc) production (as seen in FIGS. 4D and 4E), while blocking IL-4 resulted in a significant inhibition of IgE production (as seen in FIG. 4E). X5⁺ Th17 cells helped naïve B cells through IL-21, as blocking IL-21 significantly inhibited the production of IgM, IgG3, and IgA (as seen in FIG. 4D).

Thus, the capacity to induce naïve B cells to differentiate into Ig-producing cells is different among blood CD4⁺ T cell populations. Neither X5⁺ nor X5⁻ Th1 cells induce B cells to produce Igs. X5⁺ Th2 cells, but not X5⁻ Th2 cells, are capable of inducing naïve B cells to produce IgG and IgA. X5⁺ Th17 cells efficiently induce naïve B cells to produce Igs, in particular IgA, while X5⁻ Th17 cells do not show any helper activity.

X5⁺ Th subsets are altered in Juvenile dermatomyositis. The identification of functionally distinct subsets within blood X5⁺ CD4⁺ T cells revealed dysregulation of Tfh responses in autoimmune diseases. Juvenile dermatomyositis (JDM) is a chronic, multisystem autoimmune disease involving muscle, skin, gastrointestinal tract, and other organs. JDM patients with active disease typically show proximal muscle weakness and/or skin rash (Feldman et al., 2008; Suber et al., 2008). Studies on JDM have revealed several mediators common to systemic lupus erythematosus (SLE), including type I IFN (Baechler et al., 2007; Bennett et al., 2003; Blanco et al., 2001; Walsh et al., 2007). Autoantibodies can be found in JDM patients serum (Suber et al., 2008). The pathogenesis of JDM remains largely unknown.

The blood X5⁺ T cell subsets in samples from JDM patients (total 49 samples from 45 patients) and age-matched healthy pediatric controls (43 donors) were analyzed as seen in Table 1. Table 1 is below.

TABLE 1

| | JDM | | | | | | | |
|---|---|---|---|---|---|---|---|---|
| | Symptomatic | | Asymptomatic | | PSOA | | Health | |
| | Mean | Range | Mean | Range | Mean | Range | Mean | Range |
| Age | 10.1 ± 4.7 | 3-18 | 10.8 ± 4.0 | 5-18 | 8.7 4.0 | 3-17 | 10.6 ± 4.5 | 2-′18 |
| Gender | M10/F17 | | M4/F21 | | M [symbol] /F25 | | M29/F14 | |
| WBC | 7.8 ± 3.5 | 3.7-15.2 | 7.0 ± 2.0 | 4.5-11 | 0.6 ± 1.9 | 3.6-13.1 | | |
| Hgb | 12.7 ± 0.9 | 10.9-14.4 | 13.5 ± 1.0 | 11.7-15.9 | 12.8 ± 1.1 | 9.9-14.8 | | |
| Plat | 300 ± 66 | 173-424 | 308 ± 84 | 151-422 | 326 ± 88 | 194-501 | | |
| Neu# | 4.6 ± 2.7 | 1.44-10.39 | 3.7 ± 1.8 | 1.71-9.72 | 3.4 ± 1.5 | 0.9-8.7 | | |
| Lym# | 2.0 ± 0.8 | 0.4-3.47 | 2.4 ± 1.0 | 0.85-5.00 | 2.4 ± 0.7 | 1.10-4.12 | | |
| Mon# | 0.9 ± 0.5 | 0.22-2.31 | 0.6 ± 0.2 | 0.25-1.3 | 0.6 ± 0.2 | 0.17-0.91 | | |
| ESR | 17.0 ±11.7 | 3-53 | 10.7 ± 10.1 | 1-41 | 8.8 ± 6.0 | 2-25 | | |
| CPK | 1381 ± 3207 | 25-11332 | 87 ± 47 | 19-207 | 91 ± 30 | 58-162 | | |
| ALD | 6.9 ± 5.8 | 2.6->25 | 4.5 ± 1.7 | 2.5-8.5 | 4.8 ± 1.3 | 2.3-8.0 | | |
| LDH | 327 ± 249 | 126-1236 | 199 ± 45 | 127-285 | 218 ± 46 | 123-31 [symbol] | | |
| AST | 68 ± 100 | 16-389 | 22.6 ± 7.2 | 12-35 | 28 ± 9 | 11-46 | | |
| ALT | 43 ± 59 | 7-267 | 15.2 ± 6.1 | 9-35 | 19 ± 10 | 7-49 | | |

| | | | | | |
|---|---|---|---|---|---|
| CMAS | 40.0 ± 13.6 | 0-52 | 51.6 ± 1.0 | 49-52 | N/A |

| | p-value (t-test) | | | | |
|---|---|---|---|---|---|
| | JDM | JDM | JDM | JDM | JDM |
| | Symptomatic | Symptomatic | Asymptomatic | Symptomatic | Asymptomatic |
| | vs Asymptomatic | vs PSOA | vs PSOA | vs Health | vs Health |
| Age | 0.54 | 0.23 | 0.21 | [symbol] | 0.64 |
| Gender | | | | | |
| WBC | [symbol] | 0.09 | 0.40 | | |
| Hgb | 0.007 | 0.550 | [symbol] | | |
| Plat | 0.74 | 0.21 | 0.43 | | |
| Neu# | 0.19 | 0.03 | 0.44 | | |
| Lym# | 0.05 | 0.11 | 0.56 | | |
| Mon# | 0.03 | 0.002 | 0.34 | | |
| ESR | 0.05 | 0.001 | [symbol] | | |
| CPK | 0.07 | 0.12 | 0.80 | | |
| ALD | 0.07 | 0.17 | 0.51 | | |
| LDH | 0.02 | 0.02 | 0.15 | | |
| AST | 0.04 | 0.02 | 0.13 | | |
| ALT | 0.03 | 0.03 | 0.20 | | |
| CMAS | 0.0002 | | | | |

[symbol]

indicates data missing or illegible when filed

Blood samples were also obtained from age-matched pediatric patients with psoriatic arthritis (PSOA, 31 patients), a systemic inflammatory disease mediated by inflammatory T cells (Lewkowicz and Gottlieb, 2004). Thirty-five JDM patients were under standard treatment including corticosteroids, methotrexate, Etanercept (TNF antibody), and/or high-dose immunoglobulin. Some PSOA patients were under control by topical corticosteroids, methotrexate and/or Etanercept. Twenty-six samples were obtained from symptomatic JDM patients who displayed skin rash and/or muscular weakness at the sampling. These patients included three newly diagnosed patients, and a patient under no treatment.

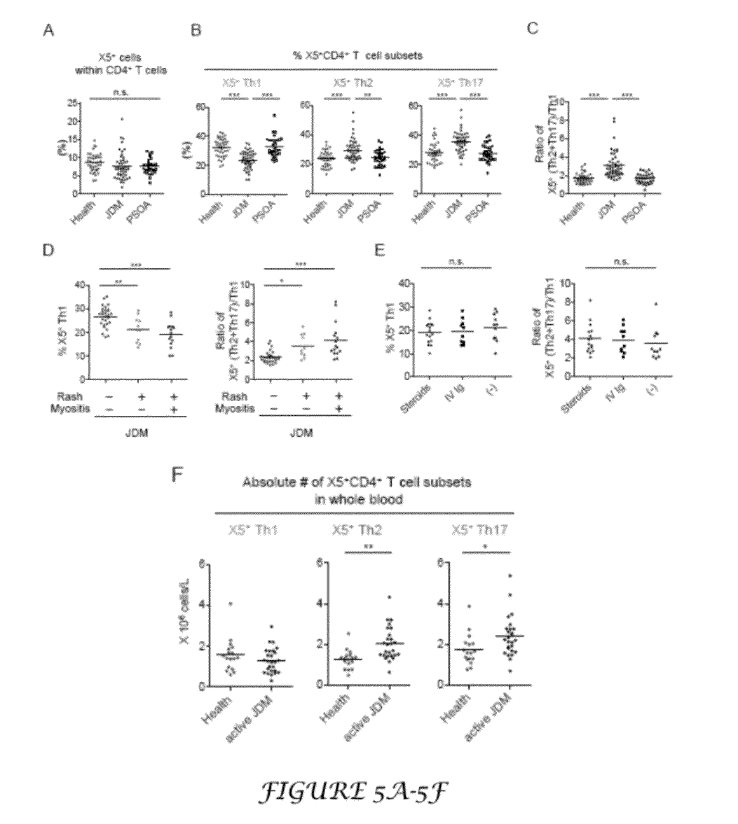

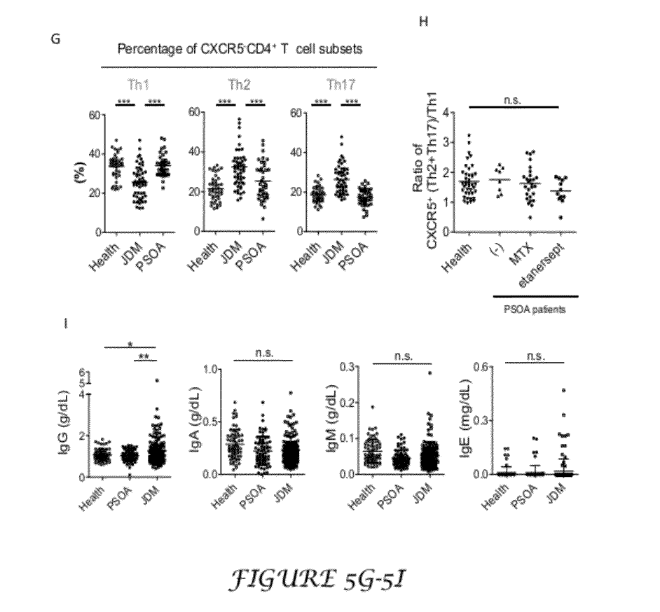

FIGS. 5A-5F are images of plots illustrating Blood X5+ Th subsets are altered in JDM. FIG. 5A is an image of a graph illustrating percentage of X5+ cells within CD4+ T cells in samples from JDM patients (n=52), PSOA patients (n=37), and age-matched healthy controls (n=41). FIG. 5B is an image of a graph illustrating percentages of the three subpopulations (X3+R6− Th1, X3−R6− Th2, X3−R6+ Th17) within blood X5+ CD4+ T cells in the three groups. One way ANOVA test, ** p<0.01, *** p<0.001. FIG. 5C is an image of a graph illustrating skewing of X5+ subsets towards Th2 and Th17 in JDM. Ratio of (X3−R6-Th2+ X3−R6+ Th17)/(X3+R6− Th1) cells within X5+ CD4+ T cells is shown. FIG. 5D is an image of a graph illustrating the frequency of X5+ Th1 cells and ratio of (Th2+Th17)/Th1 within X5+ CD4+ T cells in JDM patients with different disease activities. JDM patients were sub-grouped according to the manifestation of clinical symptoms (rash and/or myositis). Patients with high disease activity show both rash and myositis. FIG. 5E is an image of a graph illustrating the frequency of X5+ Th1 cells and ratio of (Th2+Th17)/Th1 in active JDM patients receiving different treatments. FIG. 5F is an image of a graph illustrating Absolute cell number of X5+ Th subsets in blood of active JDM patients. The cell numbers were calculated based on the complete blood cell count, lymphocyte frequency within white blood cells, and the frequency of X5+ CD4+ T cell subsets within the lymphocyte population.

The frequency of X5⁺ cells within CD4⁺ T cells was not significantly different among the three groups (JDM 7.6±0.5%, PSOA 7.6±0.3%, and control 8.6±0.4%. Mean±s.e.m. One way ANOVA test) (as illustrated in FIG. 5A). However, the frequency of Th1 (X3⁺R6⁻) cells within the X5⁺ CD4⁺ T cell compartment was significantly lower in JDM patients when compared to PSOA patients and healthy controls (as illustrated in FIG. 5B. JDM 23.5±0.8%, PSOA 32.8±1.3%, and control 32.4±1.0%. Mean±s.e.m. both p<0.0001, One way ANOVA test). In contrast, the frequencies of Th2 (X3⁻R6⁻) and Th17 (X3⁻R6⁺) cells within X5⁺ CD4⁺ T cells were higher in JDM when compared to PSOA patients and healthy controls (Th2: JDM 29.4±1.0%, PSOA 24.4±1.0%, and control 23.7±0.8%. both p<0.0001. Th17: JDM 35.8±1.0%, PSOA 27.9±1.0%, and control 28.1±1.0%. both p<0.0001). The skewing of subsets resulted in a significant increase in B helpers over non-B helpers in X5⁺ CD4⁺ T cells in JDM, as calculated by the ratio of Th2+Th17 (B helpers) over Th1 (non B-helpers) (JDM 3.1±0.2, PSOA 1.7±0.1, and control 1.7±0.1, Mean±s.e.m., both p<0.0001) (as illustrated in FIG. 5C). The Th subsets within X5⁻ CD4⁺ T cell compartment were also skewed towards Th2 and Th17 in JDM patients, suggesting the Th skewing occurs at a systemic level in JDM. Of note, in the PSOA group, patients receiving methotrexate or Etanercept showed comparable levels of X5⁺ Th subsets, indicating that these treatments did not alter the composition of X5⁺ Th subsets. Thus, blood X5⁺ Th subsets are skewed towards Th2 and Th17 in JDM patients.

Tfh subset skewing is associated with disease activity. To determine whether the skewing in Th subsets is associated with the disease activity in JDM, patients were subgrouped according to the clinical manifestations. Patients with skin rash and muscular weakness (measured by the Childhood Myositis Assessment Scale (CMAS)) showed a lower frequency of Th1 cells within X5⁺ CD4⁺ T cells (19.2±1.4%. Mean±s.e.m. n=15) than asymptomatic patients (26.7±0.8%, n=24) or patients with skin rash alone (21.2±1.6%, n=10) (as illustrated in FIG. 5D). Accordingly, patients with skin rash and muscular weakness displayed a higher ratio of Th2+Th17/Th1 in X5⁺ CD4⁺ T cells (FIG. 5D). The skewing of Th subsets is not due to the treatment, as neither the frequency of Th1 cells or the ratio of Th2+ Th17/Th1 in X5⁺ CD4⁺ T cells were different among subgroups receiving intravenous corticosteroids, high-dose immunoglobulins, or no treatment within the patients with clinical symptoms (as illustrated in FIG. 5E). Skewing in blood X5⁺ Th subsets in patients with skin rash and muscular weakness resulted in a significant increase in the absolute number of X5⁺ Th2 and X5⁺ Th17 cells, when compared to healthy controls (Th2: active JDM (n=25) 2.0±0.2 vs. Healthy (n=17) 1.3±0.1×10⁶ cells/L. Mean±s.e.m. p=0.001, t-test; Th17: active JDM 2.4±0.2 vs. Healthy 1.7±0.2×10⁶ cells/L. p=0.03) (as illustrated in FIG. 5F). Lastly, JDM patients displayed higher levels of serum IgG levels than PSOA and control groups (Figure S10. JDM: 1.27±0.67 g/dL, Mean±s.d. n=125, PSOA: 1.04±0.28 g/dL, n=73. Healthy 1.07±0.29, n=64. One-way ANOVA, both p<0.05).

FIG. 5G is an image of a plot of the CXCR5⁻ Th subsets are skewed towards Th2 and Th17 in JDM. Percentage of the within CXCR5⁻ CD4⁺ T cells in JDM patients, age-matched healthy controls, and PSOA patients. One way ANOVA test. *** p<0.001. FIG. 5H is an image of a plot of the composition of blood CXCR5⁺ Th subsets was not altered by treatments in PSOA. Ratio of Th2+Th17/Th1 in CXCR5⁺ CD4⁺ T cells in healthy controls and PSOA patients receiving different therapies is shown. One way ANOVA test. FIG. 5I is an image of a plot of JDM patients display higher levels of serum IgG. Serum Ig levels were analyzed by ELISA. One-way ANOVA. * p<0.05, ** p<0.01.

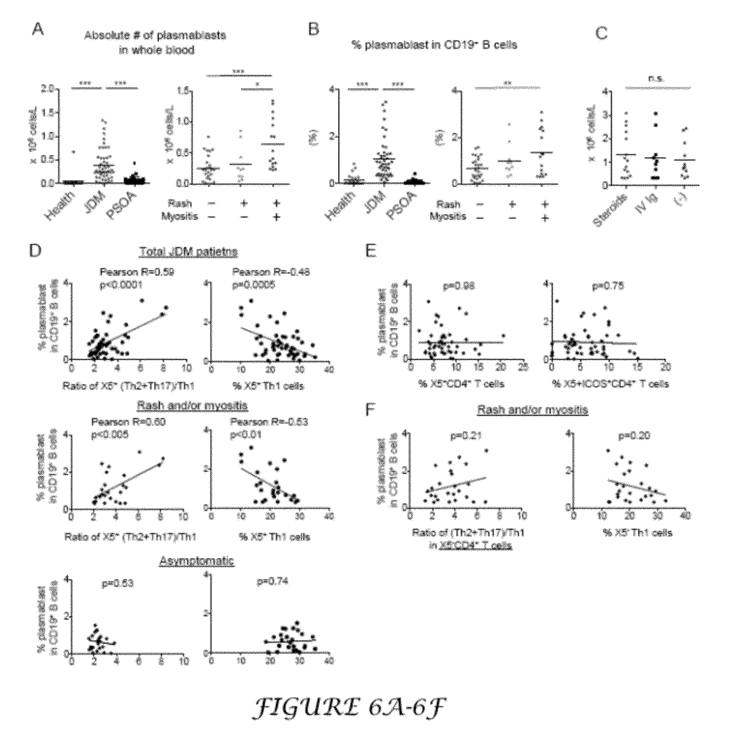

FIGS. 6A-6F are images of plots illustrating the skewing in blood X5+ CD4+ T cell subsets correlates with B cell alteration. FIG. 6A is an image of a graph illustrating the absolute numbers of plasmablasts (CD19+CD20−CD38+CD27+) in blood of JDM patients, PSOA patients, and healthy controls (left), and in JDM patients with different disease activities (right). FIG. 6B is an image of a graph illustrating the percentage of plasmablast within total CD19+ B cells. FIG. 6C is an image of a graph illustrating the absolute numbers of plasmablasts in blood of active JDM patients receiving different treatments. FIG. 6D is an image of a graph illustrating a correlation between the percentage of plasmablasts within CD19+ B cells and the ratio of X5+ (Th2+Th17)/Th1 cells (left) or the frequency of X5+ Th1 cells (right) in JDM. Pearson correlation coefficient and two-tailed p-value are shown. FIG. 6E is an image of a graph illustrating a correlation between the percentage of plasmablasts within CD19+ B cells and the frequency of X5+ CD4+ T cells (left) or of X5+ ICOS+ CD4+ T cells (right). FIG. 6C is an image of a graph illustrating Correlation between the percentage of plasmablasts and the ratio of (Th2+Th17)/Th1 within X5− CD4+ T cells (left) or the frequency of X5− Th1 cells (left) in active JDM patients.

Analysis of blood B cell subsets revealed that JDM patients displayed higher numbers of circulating plasmablasts (CD19⁺CD20⁻CD38⁺⁺ B cells (Arce et al., 2001)) than PSOA patients and controls (absolute numbers: JDM 3.9±0.5, PSOA 0.6±0.1, control 0.4±0.4×10⁵ cells/L. Mean±s.e.m., both p<0.0001. One way ANOVA test). Frequency within CD19⁺ B cells: JDM 1.0±0.13%, PSOA 0.06±0.01%, and control 0.14±0.05%. both p<0.0001) As illustrated in FIGS. 6A and 6B. Importantly, circulating plasmablasts were present at a higher number and frequency in JDM patients displaying both skin rash and muscular weakness (absolute number in blood: active JDM 6.4±1.0 vs. asymptomatic 2.5±0.4 cells×10⁵ cells/L, p<0.001; frequency within CD19⁺ B cells: active JDM 1.36±0.24% vs. asymptomatic 0.67±0.09%, p<0.01). The number of circulating plasmablasts was similar among symptomatic patients receiving different treatments, as seen in FIG. 6C.

The frequency of plasmablasts within CD19⁺ B cells positively correlated with the extent of skewing of X5⁺ CD4⁺ T cell subsets towards Th2+Th17 (as seen in FIG. 6D, top left), and negatively correlated with the frequency of X5⁺ Th1 cells (as seen in FIG. 6D, top right). The positive correlation between the frequency of plasmablasts and the skewing of X5⁺ Th subsets was limited to symptomatic patients (rash and/or muscular weakness) (as seen in FIG. 6D, middle and bottom). In contrast, the frequency of plasmablasts did not correlate with the frequency of either total X5⁺ CD4⁺ T cells (as seen in FIG. 6E, left), or ICOS⁺X5⁺CD4⁺ T cells (as seen in FIG. 6E, right). Neither the skewing of Th subsets within X5⁻ CD4⁺ T cells nor the frequency of X5⁻ Th1 cells showed a correlation with the frequency of plasmablasts (as seen in FIG. 6E).

Collectively, the alteration of the balance of blood X5⁺ Th subsets between B helpers (Th2+Th17) vs non B helpers (Th1) correlates with disease activity and with an increase in circulating plasmablasts in JDM.

Blood X5⁺ Th subsets provide biomarkers reflecting immunologic and clinical activity in human autoimmune diseases. Blood X5⁺ CD4⁺ T cells share functional properties with Tfh cells from secondary lymphoid organs. In concordance with Tfh cells, blood X5⁺ CD4⁺ T cells induced naïve and memory B cells to become Ig-producing cells via IL-21, IL-10, and ICOS, and secreted CXCL13. At variance with Tfh cells, very few blood X5⁺ CD4⁺ T cells expressed CD69 and ICOS, and low levels of PD-1 (Kim et al., 2001b; Ma et al., 2009; Simpson et al., 2010), suggesting their resting state. While X5 can be expressed by any activated CD4⁺ T cells (Schaerli et al., 2001), dissociation in the expression of X5 and activation molecules suggests that blood X5⁺ CD4⁺ T cells are not composed of recently activated cells. Consistent with the resting phenotype, blood X5⁺ CD4⁺ T cells required cell activation to provide help to B cells through cognate interaction. Furthermore, our study supports an previous observation (Rivino et al., 2004) showing that blood X5⁺ CD4⁺ T cells contained memory cells specific for microbial antigens. Blood X5⁺ CD4⁺ T cells express CCR7 and CD62L, suggesting their capacity to migrate into secondary lymphoid organs. Thus, it is plausible that upon microbial invasion, memory X5⁺ CD4⁺ T cells draining into lymphoid organs interact with B cells presenting microbial antigens, and induce their differentiation into Ig-producing cells or germinal center B cells through secretion of IL-21 (Linterman et al., 2010; MacLennan et al., 2003; Zotos et al., 2010). Blood memory X5⁺ CD4⁺ T cells might further contribute to the diversification of antibody responses through “epitope spreading”, where they interact with naïve B cells carrying a novel antigen which shares T cell epitopes, and induce B cell differentiation into Ig-producing cells (Deshmukh et al., 2007).

Human blood X5⁺ CD4⁺ T cells are composed of three subsets that can be distinguished into Type 1, Type 2, Type 17 by differential expression of CXCR3, and CCR6, and distinct cytokine profiles. Only X5⁺ Th2 and X5⁺ Th17 subsets induced naïve B cells to secrete immunoglobulins and to switch isotypes through IL-21. The two X5⁺ Th subsets promoted naïve B cells to secrete different isotypes. While X5⁺ Th2 cells promoted IgG and IgE secretion, X5⁺ Th17 cells were efficient at promoting IgG and, in particular, IgA secretion. Effector Tfh cells associated with such X5⁺ Th subsets differentially shape the quality of humoral immunity, with Type 2 cells favoring allergic response and Type 17 cells favoring protective mucosal antibody response. X5⁺ Th2 cells were previously implicated in the generation of antibody responses (Lane et al., 2005). Furthermore, recent mouse studies demonstrated the presence of IL-4-secreting X5⁺ Th2 cells in GCs (King and Mohrs, 2009; Reinhardt et al., 2009; Zaretsky et al., 2009). The difference between mouse and human Tfh cells might lie on the role of Type 1 cells. Mouse studies identified IFN-γ-secreting Type 1 Tfh cells in GCs, which promote the class-switching of GC B cells towards IgG2a (Reinhardt et al., 2009). This is contrast to human Type 1 X5⁺ CD4⁺ T cells that do not induce B cells to secrete Ig. Indeed, IFN-γ does not have any impact on isotype switching of human B cells (Banchereau et al., 1994). The actual function of human X5⁺ Th1 cells remains to be established.

Among blood X5⁻ CD4⁺ T cells, only X5⁻ Th2 cells can induce naïve B cells to become plasmablasts producing IgM and IgE. In contrast to X5⁺ Th17 cells, X5⁻ Th17 cells were completely incapable of inducing naïve B cells to secrete Igs. Neither X5⁻ Th2 nor X5⁻ Th17 cells secrete IL-21 upon interaction with naïve B cells. The molecular mechanisms whereby two different types of effectors, i.e., B-helpers and non-B-helpers, emerge from Th2 and Th17 subsets remain to be established. Multiple factors are likely involved in this process, including DC subsets that prime naive CD4⁺ T cells (Klechevsky et al., 2008), cytokines secreted by DCs (Dienz et al., 2009; Schmitt et al., 2009), or by other cell types, including IL-21 secreted by neighboring Th cells (Nurieva et al., 2008; Vogelzang et al., 2008), T cell receptor affinity against peptide/MHC complex (Fazilleau et al., 2009), and the interaction with B cells (Nurieva et al., 2008; Zaretsky et al., 2009).

Notably, consistent with a previous report (Simpson et al., 2010), the expression level of Bcl-6 mRNA was similar between blood X5⁺ CD4⁺ T cell subsets and X5⁻ CD4⁺ T cells. The expression level of Blimp-1 mRNA was lower in X5⁺ CD4⁺ T cell subsets than in X5⁻ CD4⁺ T cells. Given the reciprocal regulation between Bcl-6 and Blimp-1 in the generation of Tfh cells (Johnston et al., 2009), maintaining the expression of Blimp-1 at low levels might be a feature of memory Tfh cells. Alternatively, in concordance with B cells (Kuo et al., 2007), down regulation of Bcl-6 might be necessary for germinal center Tfh cells to become memory cells.

The alteration in the balance of Tfh subsets can be associated to autoimmunity in humans. Higher numbers of circulating plasmablasts in active JDM patients support the general consensus that JDM is an autoantibody-associated disease, and justify using JDM as a model of human autoimmune diseases. Over-representation of Th2 and Th17 subsets and under-representation of Th1 subset both within X5+ and X5− compartments suggest that the regulation of Th responses is systemically altered in JDM patients. The alteration in X5+ Th subsets displayed, however, a better correlation with the frequency of circulating plasmablasts than that in X5− Th subsets, and thus providing a better biomarker assessing the dysregulation of B cell responses. Collectively, these observations suggest that systemic dysregulation of Th development in JDM results in an over-representation of Type 2 and Type 17 Tfh cells, which promote the development of autoantibody-secreting plasma cells from an extended pool of naive precursors (Samuels et al., 2005; Yurasov et al., 2005).

The observation of three subsets within blood X5+ CD4+ T cells with different B helper functions suggests that the type of humoral responses is differentially regulated by different subsets of Tfh cells in humans, and thus are relevant to many clinical situations. For example, induction of Type 2 and Type 17, but not Type 1 cells, would be desired for efficient antibody responses to vaccination. In particular, the discovery of the Type 17 X5+ CD4+ T cell subset as a potent IgA inducer could guide the design of vaccines for protective mucosal immunity. Further characterization of blood X5+ CD4+ T cells will provide insights into the pathogenesis and perhaps identify novel therapeutic targets for human autoimmune diseases.

PBMCs were purified by Ficoll gradient centrifugation from apheresis blood samples obtained from adult healthy volunteers and kept frozen in 10% DMSO at −80° C. Fresh blood samples were collected also from JDM patients (10.8±4.2 years old, Mean±s.d. n=48) who fulfilled criteria of Bohan and Peter (Bohan and Peter, 1975), PSOA patients (7.7±4.7 years old, n=37) and age-matched pediatric controls (n=41). Patients were classified as active if they had systemic symptoms (skin rash and/or muscular weakness [measured by the Childhood Myositis Assessment Scale (CMAS), where the degree of increased muscular weakness is shown by lower scores with a cut-off value 48 of the full score 52]). Patients and pediatric controls were recruited at Texas Scottish Rite Hospital for Children in Dallas. The study was approved by the Institutional Review Boards (IRBs) of UT Southwestern Medical Center, Texas Scottish Rite Hospital, and Baylor Health Care System. Informed consent was obtained from parents or legal guardians.

CD4⁺ T cells enriched by negative selection were stained with the anti-CD4 FITC (RPA-T4), anti-CXCR5 PE (51505.111), anti-CD45RA TC (MEM-56), anti-CD14 APC (61D3), and anti-CD123 APC (AC145). Then, naïve, X5⁻ memory, and X5⁺ memory CD4⁺ T cells were sorted from APC-negative cell fractions. For X5⁺ Th subsets sorting, enriched CD4⁺CD45RA⁻ T cells were stained with anti-CCR6 biotin (11A9)+SA-TC, anti-CXCR3 FITC (49801), anti-CXCR5 PE, and anti-CD4 APC. Positively selected CD19⁺ B cells were stained with anti-IgD FITC (IA6-2), anti-CD27 PE (L128), and anti-CD3 APC. Naïve and memory B cells were sorted as IgD+CD27−CD3− and CD27⁺CD3−CD19⁺ cells, respectively.

CD4⁺ T cell and B cell Co-culture. Naïve B cells pre-treated with sepharose-conjugated rabbit anti-human IgM Ab (1 μg/ml) were co-cultured with sorted CD4⁺ T cells (2×10⁴ cells each/well. 5×10⁴ cells each/well for cytokine measurement) in the presence of endotoxin-reduced SEB (1 μg/ml) in RPMI 1640 complete medium supplemented with 10% heat-inactivated FBS. Cytokine levels were measured in the culture supernatants at day 2 by Luminex, and the Ig levels were measured at day 12 by ELISA. In some instances, IL-21R/Fc (20 μg/ml, R&D), ICOS-L-mIgFc (15 μg/ml; Ancell), anti-IL-4 mAb (20 μg/ml, MP4-25D2), or anti-IL-10 mAb (25 μg/ml, developed and validated at the Institute) were added to the cultures.

The concentrations of IL-4, IL-5, IL-13, IL-17A, IL-21 (Schmitt et al., 2009), and IFN-γ were determined by a bead-based multiplex cytokine assay (LUMINEX™). Mouse monoclonal antibodies specific for IL-5, IL-13, IL-17A, and IL-21 were generated in the Institute, and conjugated to microbeads. Antibody pairs for cytokine measurement were validated for the measurement by LUMINEX. The concentrations of IL-22 were determined with ELISA.

Total RNA was extracted from sorted CD4⁺ T cells with an RNeasy Mini Kit (Qiagen) according to the manufacture's instruction. Single-strand cDNA was synthesized with Superscript reverse transcriptase III and oligo(dT) primers (Invitrogen). PCR was performed in 35 cycles for transcription factors and in 25 cycles for β-actin with the following primer pairs. T-bet; forward SEQ ID No: 1: 5′-CACTACAGGATGTTTGTGGACGTG-3′ and reverse SEQ ID No: 2: 5′-CCCCTTGTTGTTTGTGAGCTTTAG-3′; GATA-3; forward SEQ ID No: 3: 5′-TGT CTGCAGCCAGGAGAGC-3′; and reverse SEQ ID No: 4: 5′-ATGCATCAAACAACTGTGGCCA-3′; RORγt; forward SEQ ID No: 5: 5′-TCTGGAGCTGGCCTTTCATCATCA-3′; and reverse SEQ ID No: 6: 5′-TCTGCTCACTTCCAAAGAGCTGGT-3′; β-actin; forward SEQ ID No: 7: 5′-TCCTGTGGCATCCACGAAACT-3′; and reverse SEQ ID No: 8: 5′-GAAGCATTTGCGGTGGACGAT-3′. PCR products were separated through 1% agarose gel, stained with ethidium bromide and visualized with an UV transilluminator.

Total RNA was extracted from blood Th subpopulations or cultured B cells using RNeasy mini kit (QIAGEN), and reverse-transcribed into cDNA in a 96-well plate using the High Capacity cDNA Archive kit (Applied Biosystems). The primer pairs (Integrated DNA Technology) used in this study was designed using the Roche Primer Design Program. Primer sequences were as follows: Bcl-6 (Accession number: NM_{—}001706.2) forward primer: SEQ ID No: 9: 5′ TTCCGCTACAAGGGCAAC-3′; reverse primer: SEQ ID No: 10: 5′-TGCAACGATAGGGTTTCTCA-3′; Prdm1 (Accession number: NM_{—}001198.2) forward primer: SEQ ID No: 11: 5′ GTGGTGGGTTAATCGGTTTG-3′; reverse primer: SEQ ID No: 12: 5′-GAAGCTCCCCTCTGGAATAGA-3′; and AICDA (Accession number: NM_{—}020661.2) forward primer: SEQ ID No: 13: 5′ GACTTTGGTTATCTTCGCAATAAGA-3′; reverse primer: SEQ ID No: 14: 5′-AGGTCCCAGTCCGAGATGTA-3′.

Real-time PCR was set up with Roche Probes Master reagents and Universal Probe Library hydrolysis probes. PCR reaction was performed on the LIGHTCYCLER 480 (Roche Applied Science) followed these conditions: step 1 (denaturation) at 95° C. for 5 min, step 2 (amplification) at 60° C. for 30 min, step 3 (cooling) at 40° C. for 30 seconds. The expression level of each gene was normalized to the levels of housekeeping gene HRPT1.

Whole blood cells were stained with these mAbs: CXCR5-Alexa488 (RF8B2), CCR6-PE (11A9), CD45RA-ECD (2H4LDH11LDB9), CXCR3-PECy5 (106), CD3-AF700 (UCHT1), CD4-Pacific Blue (RPA-T4), CD45-Pacific Orange (HI30), CD19-ECD (J3.119), CD20-PECy5 (2H7), CD38-PECy7 (HB7), CD27-APCH7 (O323). The stained cells were analyzed with BD LSRII.

The significance of the difference between groups was analyzed with One way ANOVA test with Bonferroni correction. The significance of the difference between two groups was evaluated by F-test followed by the two-tailed Student's, or paired t-test. A Wilcoxon signed-ranks test was applied when data did not show Gaussian distribution. Pearson correlation coefficient and two-tailed p-value were determined in the analysis of correlations. A value of p<0.05 was considered significant.

### Example 2

### IL-6 Promotes Tfh Development in Humans and Anti-IL-6 can be Used to Target Autoimmune Diseases

Development of specific antibody response requires help by CD4+ T cells primed by dendritic cells (DCs). In particular, T follicular helper cells (Tfh) represent a CD4+ T cell subset specialized for the help of B cells via the secretion of Interleukin (IL)-21 (Fazilleau et al., 2009; King et al., 2008; Vinuesa et al., 2005). The present inventors have reported that human DCs induce IL-21-producing Tfh-like cells through IL-12 (Schmitt et al., 2009). The present inventors found, surprisingly, that IL-6 further promotes the development of Tfh cells in collaboration with IL-12, in particularly in the presence of Transforming Growth Factor-beta (TGF-b). The combination of IL-6 and TGF-b strongly suppresses the development of Th1 cells in response to stimulation with IL-12, while it potently promotes the development of Tfh cells. Our discovery suggests that IL-6 blocking is beneficial for the treatment of human autoimmune diseases, where over-representation of Tfh cells leads to the generation of autoantibodies.

As stated hereinabove Tfh cells represent a subset of CD4+ T cells, which express chemokine (C-X-C motif) receptor 5 (CXCR5), and are specialized for B cell help in germinal centers (GCs). The present inventors in U.S. Patent Application Ser. No. 61/431,986 (relevant portions incorporated herein by reference) have previously shown that human blood CXCR5+CD4+ T cells largely share the functional properties with Tfh cells, and represent their circulating memory compartment. Blood CXCR5+CD4+ T cells comprise three subsets, Th1, Th2 and Th17 cells, which can be distinguished based on chemokine receptor expression. Th2 and Th17 cells within CXCR5+, but not CXCR5−, compartment efficiently induce naïve B cells to produce immunoglobulins via Interleukin (IL)-21. CXCR5+ Th17 cells were potent at inducing IgA response. In contrast, Th1 cells, from both CXCR5+ and CXCR5− compartments, lack the capacity to help B cells. Patients with juvenile dermatomyositis (JDM), a systemic autoimmune disease, displayed a profound skewing of blood CXCR5+CD4+ T cell subsets towards Th2 and Th17. Significantly, the skewing of subsets correlated with disease activity and frequency of blood plasmablasts. Collectively, these observations suggested that an altered balance of Tfh subsets contributes to human autoimmunity.

U.S. Patent Application Ser. No. 61/431,986 provides evidence that human blood CXCR5+CD4+ T cells represent a circulating pool of memory Tfh cells. Significantly, blood CXCR5+ CD4+ T cells can be distinguished into Th1, Th2, and Th17 populations according to chemokine receptor expression and by the type of cytokine secretion patterns. These subsets differentially regulate the differentiation and class-switching of naïve B cells. Finally, an alteration of blood CXCR5+ CD4+ T cell subsets in an autoimmune disease, JDM is also disclosed in the '986 invention.

The present inventors have shown that juvenile dermatomyositis (JD) patients' blood CXCR5+CD4+ T cell subsets are skewed towards Th2 and Th17 cells. PBMCs were purified from apheresis blood samples obtained from adult healthy volunteers to be used in the studies. Fresh blood samples were also collected from JDM (n=52), PSOA patients (n=31) and age-matched pediatric controls (n=43).

Whole blood cells were stained with these mAbs: CXCR5-Alexa488 (RF8B2), CCR6-PE (11A9), CD45RA-ECD (2H4LDH11LDB9), CXCR3-PECy5 (106), CD3-AF700 (UCHT1), CD4-Pacific Blue (RPA-T4), CD45-Pacific Orange (HI30), CD19-ECD (J3.119), CD20-PECy5 (2H7), CD38-PECy7 (HB7), CD27-APCH7 (O323). The stained cells were analyzed with BD LSRII.

The significance of the difference between groups was analyzed with One way ANOVA test with Bonferroni correction. The significance of the difference between two groups was evaluated by F-test followed by the two-tailed Student's, or paired t-test. A Wilcoxon signed-ranks test was applied when data did not show Gaussian distribution. Pearson correlation coefficient and two-tailed p-value were determined in the analysis of correlations.

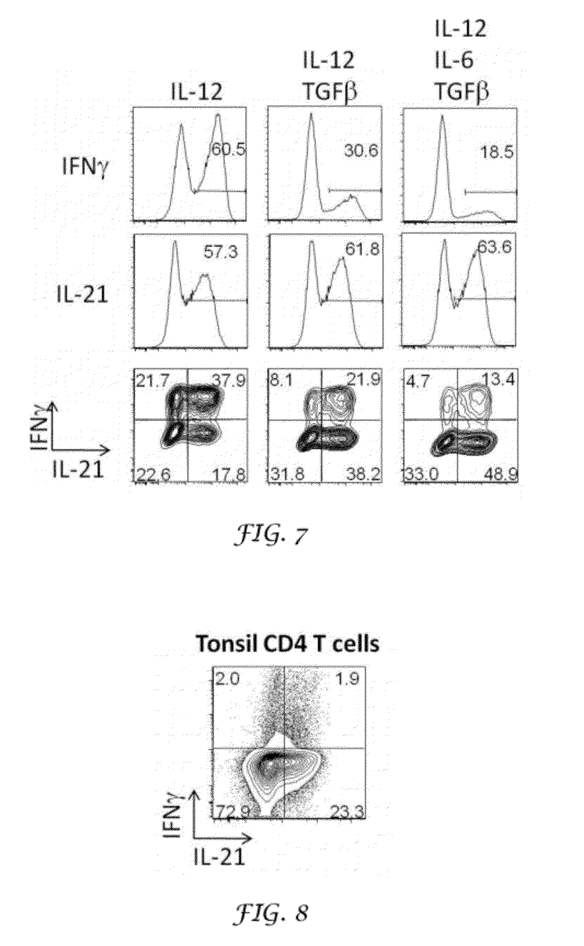

The present inventors further analyzed the developmental pathway of human Tfh cells in vitro. Naïve CD4+ T cells were stimulated for 7 d with anti-CD3/CD28 mAbs in the presence of different sets of cytokines. First, the inventors examined intracytoplasmic cytokine expression by the cultured CD4+ T cells upon re-stimulation with PMA/ionomycin. As shown in FIG. 7, TGF-β together with either IL-12 alone or the combination of IL-12 and IL-6 promotes the development of IFN-γ-IL-21+ CD4+ T cells. PBMCs were purified from apheresis blood samples obtained from adult volunteers. CD4+ T cells were first enriched by negative selection with purified CD8 (HIT8a), CD11b (LM1/2), CD11c (B-ly6), CD14 (M5E2), CD15 (W6D3), CD16 (3G8), CD19 (J4.119), CD45RO (UCHL1), CD56 (C218) and HLA-DR (B8.12.2) mAbs, and Dynabeads Pan Mouse IgG (Dynal). Naive CD4+ T cells were further purified by sorting with FACSAria (BD Biosciences) as CCR7+ CD8− CD56-HLA-DR-CD45RA+CD4+ cells. Cell purity was >99%.

After overnight stimulation of naïve CD4+ T cells (5×10⁶ cells/ml) with CD3/CD28 Dynabeads (Invitrogen), cells were recovered and cultured with plate-bound CD3 mAb (5 μg/ml, OKT3) and soluble CD28 mAb (1 μg/ml, CD28.2) in flat bottom 96 well plates in RPMI complete medium in the presence of: IL-6 (10 ng/ml), IL-12 (1 or 10 ng/ml) and TGF-β (5 ng/ml) (all from R&D).

Naïve CD4+ T cells stimulated for 4 d were harvested and stimulated with PMA (25 ng/ml) and ionomycin (1 μg/ml) for 6 h in the presence of GolgiStop (BD Biosciences) and Brefeldine for the last 4 h. Cells were then stained as needed with CD3 (S4.1) and CD8 (SFCI21Thy2D3), fixed and permeabilized and the expressed cytokines in the cytoplasm were analyzed with IL-21 (3A3-N2) and IFN-γ (4S.B3) mAbs. Dead cells were further excluded from the analysis by labeling with cells LIVE/DEAD fixable Aqua (Invitrogen). Cells were acquired on a BD FACS Canto II or a BD LSRII. Expression of each molecule was assessed in activated CD4+ T cells (FSChighCD3+CD8− cells) with FlowJo software (TreeStar).

In contrast, the combination of IL-6/TGF-β/IL-12 induced naïve CD4+ T cells to express IL-21 at similar levels with IL-12 alone. Accordingly, the combination of IL-6/TGF-β/IL-12 yielded more CD4+ T cells expressing IL-21 but not IFN-γ, a cytokine expression profile of human tonsillar Tfh cells (FIG. 8).

Tonsil samples were obtained from young patients (3-12 years) undergoing tonsillectomy, and single cells were collected by mechanical disruption of tonsil samples. B cells were removed with CD19 MACS Microbeads (Miltenyi Biotech). Tonsilar T cells were stimulated with PMA (25 ng/ml) and ionomycin (1 μg/ml) for 6 h in the presence of GolgiStop (BD Biosciences) and Brefeldine for the last 4 h. Cells were then stained as needed with CD3 (S4.1) and CD8 (SFCI21Thy2D3), fixed and permeabilized and the expressed cytokines in the cytoplasm were analyzed with IL-21 (3A3-N2) and IFN-γ (4S.B3) mAbs. Dead cells were further excluded from the analysis by labeling with cells LIVE/DEAD fixable Aqua (Invitrogen). Cells were acquired on a BD FACS Canto II or a BD LSRII. Expression of each molecule was assessed in CD4+ T cells (CD3+CD8− cells) with FlowJo software (TreeStar). From FIG. 8 it can be seen that a large fraction of tonsillar CD4+ T cells express IL-21 but not IFN-γ.

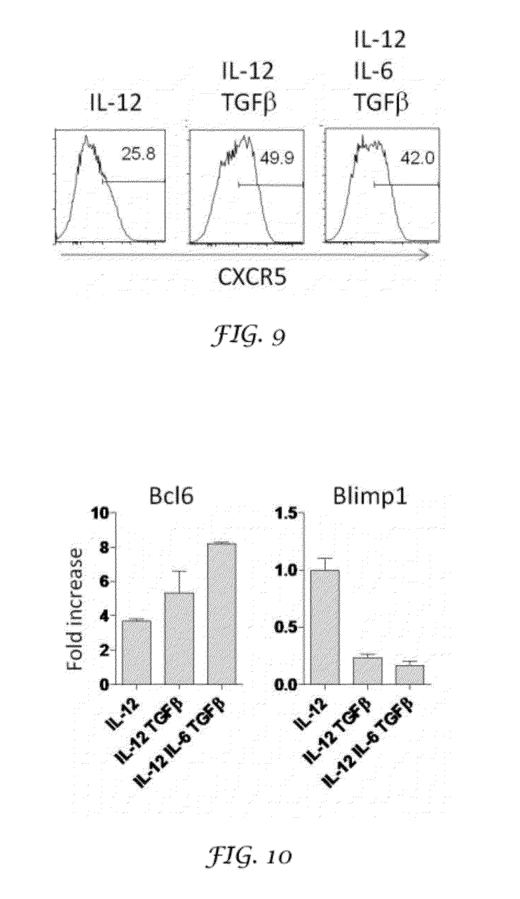

Human Tfh cells express the chemokine receptor, CXCR5, which plays a central role for their migration into B cell follicles (Breitfeld et al., 2000; Kim et al., 2001; Schaerli et al., 2000). The inventors analyzed the expression of CXCR5 on the stimulated CD4+ T cells. As shown in FIG. 9, CD4+ T cells stimulated with the combination of IL-6/TGF-b/IL-12 expressed higher levels of CXCR5 than those stimulated with IL-12 alone. After overnight stimulation of naïve CD4+ T cells (5×10⁶ cells/ml) with CD3/CD28 Dynabeads (Invitrogen), cells were recovered and cultured with plate-bound CD3 mAb (5 μg/ml, OKT3) and soluble CD28 mAb (1 μg/ml. CD28.2) in flat bottom 96 well plates in RPMI complete medium in the presence of: IL-6 (10 ng/ml), IL-12 (1 or 10 ng/ml) and TGF-β (5 ng/ml) (all from R&D). Naïve CD4+ T cells stimulated for 4 d were harvested and stained with CXCR5 (RF8B2) mAb. Thus, TGF-β together with either IL-12 alone or the combination of IL-12 and IL-6 promotes the expression of CXCR5 on activated CD4+ T cells.

The development of Tfh cells is regulated by the balance of two transcription repressors, Bcl-6 and Blimp-1. While Bcl-6 is essential and sufficient in the development of Tfh cells, Blimp-1 suppresses their development (Johnston et al., 2009; Nurieva et al., 2009; Yu et al., 2009). The inventors analyzed the expression levels of Bcl-6 and Blimp-1 in the stimulated CD4+ T cells by real-time RT-PCR. As shown in FIG. 10, CD4+ T cells stimulated with the combination of IL-6/TGF-b/IL-12 expressed higher levels of Bcl-6 and lower levels of Blimp-1 than those stimulated with IL-12 alone or the combination of IL-12/TGF-b. This observation shows that IL-6 together with IL-12/TGF-b skews the differentiation of CD4+ T cells towards Tfh lineage.

After overnight stimulation of naïve CD4+ T cells (5×10⁶ cells/ml) with CD3/CD28 Dynabeads (Invitrogen), cells were recovered and cultured with plate-bound CD3 mAb (5 μg/ml, OKT3) and soluble CD28 mAb (1 μg/ml, CD28.2) in flat bottom 96 well plates in RPMI complete medium in the presence of: IL-6 (long/ml), IL-12 (1 or 10 ng/ml) and TGF-β (5 ng/ml) (all from R&D).

Naïve CD4 T cells cultured for 2 days were lysed in RLT buffer. Total RNA was purified using RNeasy Micro Kit (Qiagen) and reverse-transcribed into cDNA in a 96-well plate using the High Capacity cDNA reverse transcription kit (Applied Biosystems). The primers and probes used were purchased from Applied Biosystems (BCL6: Hs00277037, PRDM1: Hs00153357 and ACTB: Hs99999903). PCR reactions were performed on the LightCycler 480 (Roche Applied Science) followed these conditions: step 1 (denaturation) at 95° C. for 5 min, step 2 (amplification for 45 cycles) at 95° C. for 10 s and 60° C. for 30 s, and step 3 (cooling) at 40° C. for 30 s. The expression of each gene was normalized to that of the housekeeping gene ACTB. Thus, as seen in FIG. 10 the combination of IL-6, IL-12, and TGF-β induces CD4+ T cells to express high levels of Bcl-6 and low levels of Blimp-1

Finally, the function to help B cells was analyzed. Stimulated CD4+ T cells were co-cultured with autologous B cells for 12 d, and the produced immunoglobulin levels were determined by ELISA. As shown in FIG. 11 naïve CD4+ T cells cultured with the combination of IL-6, IL-12, and TGF-β efficiently induce B cells to produce immunoglobulins than those stimulated with IL-12 alone or the combination of IL-12 and TGF-β. Thus, CD4+ T cells stimulated with IL-6/TGF-β/IL-12 acquire the capacity to efficiently help B cells.

After overnight stimulation of naïve CD4+ T cells (5×10⁶ cells/ml) with CD3/CD28 Dynabeads (Invitrogen), cells were recovered and cultured with plate-bound CD3 mAb (5 μg/ml, OKT3) and soluble CD28 mAb (1 μg/ml, CD28.2) in flat bottom 96 well plates in RPMI complete medium in the presence of: IL-6 (10 ng/ml), IL-12 (1 or 10 ng/ml) and TGF-β (5 ng/ml) (all from R&D).

B cells were first enriched from the same donor's PBMCs by positive selection using CD19 MicroBeads (Miltenyi Biotec). Memory B cells were sorted with FACSAria as CD27+ CD3− CD11c− CD14− cells, respectively. Cell purity was >98%.

Activated naïve CD4+ T cells were sorted at day 4 as FSChigh cells Activated CD4+ T cells were co-cultured with autologous memory B cells (4×10⁴ cells/well each) in 96-well round-bottom plates in Yssel medium/10% FBS in the presence of endotoxin-reduced SEB (0.25 ng/ml; Toxin technology). IgG produced in the cultures were analyzed by ELISA at day 6.

Collectively, IL-6 in collaboration with IL-12/TGF-b induces naïve CD4+ T cells to: (1) express IL-21 but IFN-g, (2) express CXCR5, (3) express Bcl-6, but not Blimp-1, and (4) acquire the capacity to help B cells. Thus, IL-6 positively regulates Tfh development in humans.

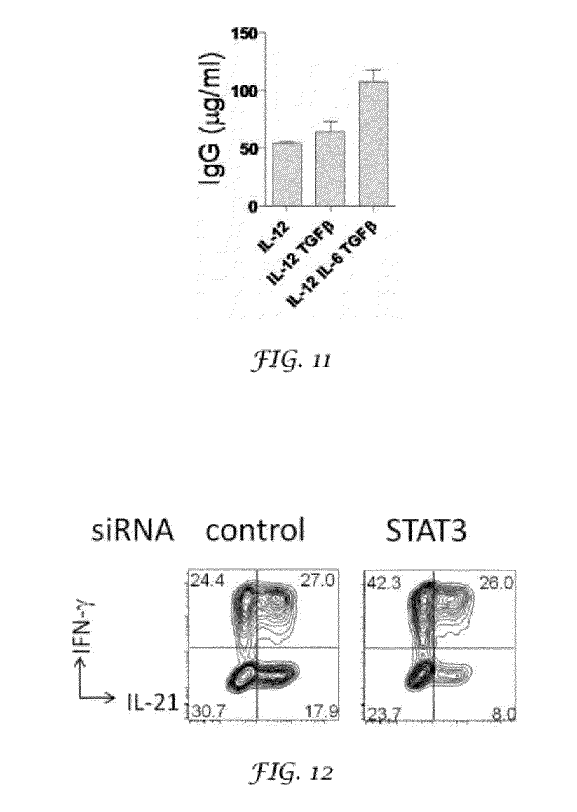

IL-6 delivers activation signals through STAT3. To examine whether the positive regulation of Tfh development by IL-6 is dependent on STAT3, small-interfering RNA (siRNA) specific for STAT3 was transfected to naïve CD4+ T cells to block STAT3 expression. Transfected cells were stimulated with the combination of IL-6/TGF-b/IL-12. As shown in FIG. 12, STAT3 siRNA transfection resulted in the strong inhibition of the development of IFN-γ-IL-21+ cells, while promoting IFN-γ+IL-21− Th1 cells. Thus, IL-6 promotes the development of IFN-γ-IL-21+ Tfh cells in a STAT3-dependent manner.

After overnight stimulation of naïve CD4+ T cells with CD3/CD28 Dynabeads, cells were transfected with siRNA using the Human T cell Nucleofector Kit and Nucleofector II device (Amaxa) according to manufacturer's instructions. siRNA to target STAT3 (s743) and silencer select negative control #1 siRNA (Ambion) were used at 5 μM (0.5 nmol/5×10⁶ cells/transfection). Cells were transferred at 6 h post-transfection to the wells with CD3/CD28 Abs. The combination of IL-6, IL-12, and TGF-β was added to the culture 24 h post transfection. Naïve CD4+ T cells cultured for 2 days with the combination of cytokines were harvested and stimulated with PMA (25 ng/ml) and ionomycin (1 μg/ml) for 6 h in the presence of GolgiStop (BD Biosciences) and Brefeldine for the last 4 h. Cells were then stained as needed with CD3 (S4.1) and CD8 (SFCI21Thy2D3), fixed and permeabilized and the expressed cytokines in the cytoplasm were analyzed with IL-21 (3A3-N2) and IFN-γ (4S.B3) mAbs. Dead cells were further excluded from the analysis by labeling with cells LIVE/DEAD fixable Aqua (Invitrogen). Cells were acquired on a BD FACS Canto II or a BD LSRII. Expression of each molecule was assessed in activated CD4+ T cells (FSChighCD3+CD8− cells) with FlowJo software (TreeStar). Thus, the combination of IL-6, IL-12, and TGF-β induces CD4+ T cells to express IL-21 through STAT3.

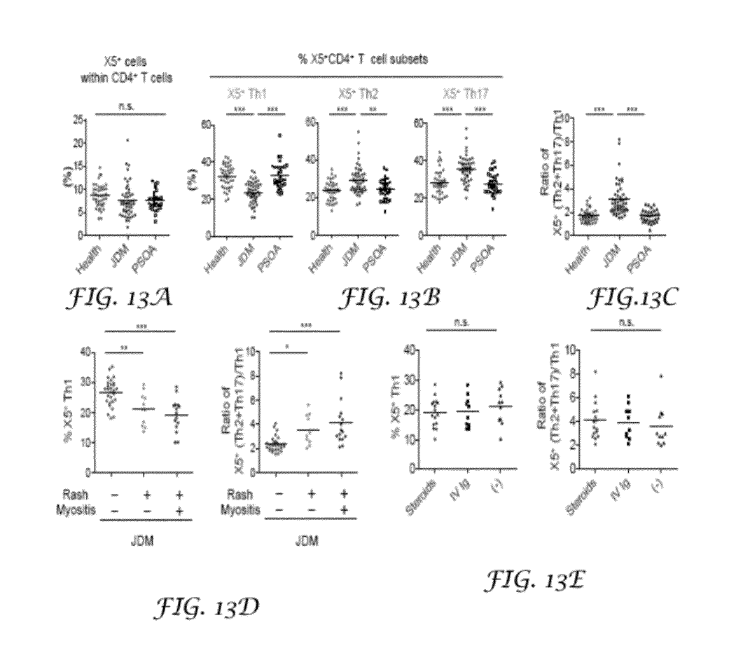

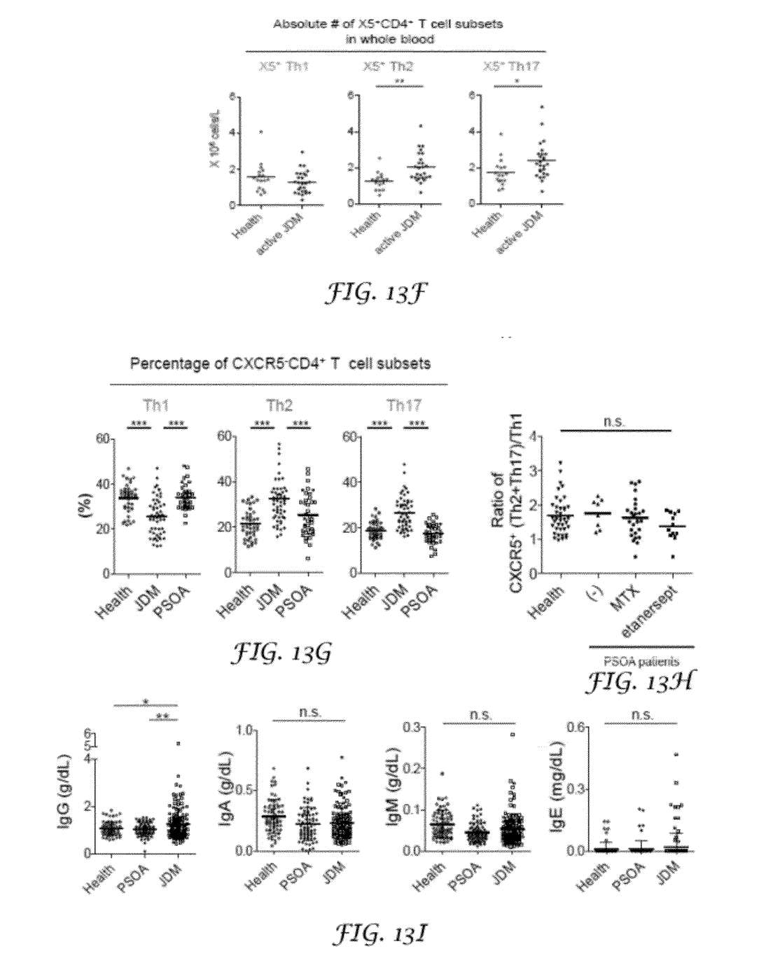

The frequency of X5+ cells within CD4+ T cells was not significantly different among the three groups (JDM 7.6±0.5%, PSOA 7.6±0.3%, and control 8.6±0.4%. Mean±s.e.m. One way ANOVA test) (as illustrated in FIG. 13A). However, the frequency of Th1 (X3+R6−) cells within the X5+ CD4+ T cell compartment was significantly lower in JDM patients when compared to PSOA patients and healthy controls (as illustrated in FIG. 13B. JDM 23.5±0.8%, PSOA 32.8±1.3%, and control 32.4±1.0%. Mean±s.e.m. both p<0.0001, One way ANOVA test). In contrast, the frequencies of Th2 (X3−R6−) and Th17 (X3−R6+) cells within X5+ CD4+ T cells were higher in JDM when compared to PSOA patients and healthy controls (Th2: JDM 29.4±1.0%, PSOA 24.4±1.0%, and control 23.7±0.8%. both p<0.0001. Th17: JDM 35.8±1.0%, PSOA 27.9±1.0%, and control 28.1±1.0%. both p<0.0001). The skewing of subsets resulted in a significant increase in B helpers over non-B helpers in X5+ CD4+ T cells in JDM, as calculated by the ratio of Th2+Th17 (B helpers) over Th1 (non B-helpers) (JDM 3.1±0.2, PSOA 1.7±0.1, and control 1.7±0.1, Mean±s.e.m., both p<0.0001) (as illustrated in FIG. 13C). The Th subsets within X5− CD4+ T cell compartment were also skewed towards Th2 and Th17 in JDM patients, suggesting the Th skewing occurs at a systemic level in JDM. Of note, in the PSOA group, patients receiving methotrexate or Etanercept showed comparable levels of X5+ Th subsets, indicating that these treatments did not alter the composition of X5+ Th subsets. Thus, blood X5+ Th subsets are skewed towards Th2 and Th17 in JDM patients.

Tfh subsets skewing is associated with disease activity. To determine whether the skewing in Th subsets is associated with the disease activity in JDM, patients were sub-grouped according to the clinical manifestations. Patients with skin rash and muscular weakness (measured by the Childhood Myositis Assessment Scale (CMAS)) showed a lower frequency of Th1 cells within X5+ CD4+ T cells (19.2±1.4%. Mean±s.e.m. n=15) than asymptomatic patients (26.7±0.8%, n=24) or patients with skin rash alone (21.2±1.6%, n=10) (as illustrated in FIG. 13D). Accordingly, patients with skin rash and muscular weakness displayed a higher ratio of Th2+Th17/Th1 in X5+ CD4+ T cells (FIG. 13D). The skewing of Th subsets is not due to the treatment, as neither the frequency of Th1 cells or the ratio of Th2+Th17/Th1 in X5+ CD4+ T cells were different among subgroups receiving intravenous corticosteroids, high-dose immunoglobulins, or no treatment within the patients with clinical symptoms (as illustrated in FIG. 13E). Skewing in blood X5+ Th subsets in patients with skin rash and muscular weakness resulted in a significant increase in the absolute number of X5+ Th2 and X5+ Th17 cells, when compared to healthy controls (Th2: active JDM (n=25) 2.0±0.2 vs. Healthy (n=17) 1.3±0.1×10⁶ cells/L. Mean±s.e.m. p=0.001, t-test; Th17: active JDM 2.4±0.2 vs. Healthy 1.7±0.2×10⁶ cells/L. p=0.03) (as illustrated in FIG. 13F). Lastly, JDM patients displayed higher levels of serum IgG levels than PSOA and control groups (JDM: 1.27±0.67 g/dL, Mean±s.d. n=125, PSOA: 1.04±0.28 g/dL, n=73. Healthy 1.07±0.29, n=64. One-way ANOVA, both p<0.05).

FIG. 13G is an image of a plot of the CXCR5− Th subsets are skewed towards Th2 and Th17 in JDM. Percentage of the within CXCR5− CD4+ T cells in JDM patients, age-matched healthy controls, and PSOA patients. One way ANOVA test. *** p<0.001. FIG. 13H is an image of a plot of the composition of blood CXCR5+ Th subsets was not altered by treatments in PSOA. Ratio of Th2+Th17/Th1 in CXCR5+ CD4+ T cells in healthy controls and PSOA patients receiving different therapies is shown. One way ANOVA test. FIG. 13I is an image of a plot of JDM patients display higher levels of serum IgG. Serum Ig levels were analyzed by ELISA. One-way ANOVA. * p<0.05, ** p<0.01.

The findings of the present invention demonstrate that IL-6 promotes Tfh development in humans and show that IL-6 is a candidate of targets in autoimmune disease, where autoantibodies play pathological roles. Thus, any composition that blocks the in vivo function of IL-6 can be applied for the treatment of autoimmune diseases, e.g., those taught in U.S. Pat. No. 7,820,790, relevant portions incorporated herein by reference. Examples of IL-6 antagonists are taught in, e.g., U.S. Patent Application Publication No. 2007/0178098, relevant portions incorporated herein by reference.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

### Example 1

Acosta-Rodriguez, E. V., Rivino, L., Geginat, J., Jarrossay, D., Gattorno, M., Lanzavecchia, A., Sallusto, F., and Napolitani, G. (2007). Surface phenotype and antigenic specificity of human interleukin 17-producing T helper memory cells. Nature immunology 8, 639-646.
Allen, C. D., Okada, T., and Cyster, J. G. (2007). Germinal-center organization and cellular dynamics. Immunity 27, 190-202.
Annunziato, F., Cosmi, L., Santarlasci, V., Maggi, L., Liotta, F., Mazzinghi, B., Parente, E., Fili, L., Ferri, S., Frosali, F., et al. (2007). Phenotypic and functional features of human Th17 cells. The Journal of experimental medicine 204, 1849-1861.
Ansel, K. M., McHeyzer-Williams, L. J., Ngo, V. N., McHeyzer-Williams, M. G., and Cyster, J. G. (1999). In vivo-activated CD4 T cells upregulate CXC chemokine receptor 5 and reprogram their response to lymphoid chemokines. The Journal of experimental medicine 190, 1123-1134.
Arce, E., Jackson, D. G., Gill, M. A., Bennett, L. B., Banchereau, J., and Pascual, V. (2001). Increased frequency of pre-germinal center b cells and plasma cell precursors in the blood of children with systemic lupus erythematosus. J Immunol 167, 2361-2369.
Baechler, E. C., Bauer, J. W., Slattery, C. A., Ortmann, W. A., Espe, K. J., Novitzke, J., Ytterberg, S. R., Gregersen, P. K., Behrens, T. W., and Reed, A. M. (2007). An interferon signature in the peripheral blood of dermatomyositis patients is associated with disease activity. Mol Med 13, 59-68.
Banchereau, J., Bazan, F., Blanchard, D., Briere, F., Galizzi, J. P., van Kooten, C., Liu, Y. J., Rousset, F., and Saeland, S. (1994). The CD40 antigen and its ligand. Annual review of immunology 12, 881-922.
Bauquet, A. T., Jin, H., Paterson, A. M., Mitsdoerffer, M., Ho, I. C., Sharpe, A. H., and Kuchroo, V. K. (2009). The costimulatory molecule ICOS regulates the expression of c-Maf and IL-21 in the development of follicular T helper cells and TH-17 cells. Nature immunology 10, 167-175.
Bennett, L., Palucka, A. K., Arce, E., Cantrell, V., Borvak, J., Banchereau, J., and Pascual, V. (2003). Interferon and granulopoiesis signatures in systemic lupus erythematosus blood. The Journal of experimental medicine 197, 711-723.
Blanco, P., Palucka, A. K., Gill, M., Pascual, V., and Banchereau, J. (2001). Induction of dendritic cell differentiation by IFN-alpha in systemic lupus erythematosus. Science (New York, N.Y. 294, 1540-1543.
Bohan, A., and Peter, J. B. (1975). Polymyositis and dermatomyositis (first of two parts). The New England journal of medicine 292, 344-347.
Bonecchi, R., Bianchi, G., Bordignon, P. P., D'Ambrosio, D., Lang, R., Borsatti, A., Sozzani, S., Allavena, P., Gray, P. A., Mantovani, A., and Sinigaglia, F. (1998). Differential expression of chemokine receptors and chemotactic responsiveness of type 1 T helper cells (This) and Th2s. The Journal of experimental medicine 187, 129-134.
Bossaller, L., Burger, J., Draeger, R., Grimbacher, B., Knoth, R., Plebani, A., Durandy, A., Baumann, U., Schlesier, M., Welcher, A. A., et al. (2006). ICOS Deficiency Is Associated with a Severe Reduction of CXCR5+CD4 Germinal Center Th Cells. J Immunol 177, 4927-4932.
Breitfeld, D., Ohl, L., Kremmer, E., Ellwart, J., Sallusto, F., Lipp, M., and Forster, R. (2000). Follicular B helper T cells express CXC chemokine receptor 5, localize to B cell follicles, and support immunoglobulin production. The Journal of experimental medicine 192, 1545-1552.
Bryant, V. L., Ma, C. S., Avery, D. T., Li, Y., Good, K. L., Corcoran, L. M., de Waal Malefyt, R., and Tangye, S. G. (2007). Cytokine-mediated regulation of human B cell differentiation into Ig-secreting cells: predominant role of IL-21 produced by CXCR5+ T follicular helper cells. J Immunol 179, 8180-8190.
Chattopadhyay, P. K., Yu, J., and Roederer, M. (2005). A live-cell assay to detect antigen-specific CD4+ T cells with diverse cytokine profiles. Nat Med 11, 1113-1117.
Chtanova, T., Tangye, S. G., Newton, R., Frank, N., Hodge, M. R., Rolph, M. S., and Mackay, C. R. (2004). T follicular helper cells express a distinctive transcriptional profile, reflecting their role as non-Th1/Th2 effector cells that provide help for B cells. J Immunol 173, 68-78.
Clark, E. A., and Ledbetter, J. A. (1994). How B and T cells talk to each other. Nature 367, 425-428.
Dent, A. L., Shaffer, A. L., Yu, X., Allman, D., and Staudt, L. M. (1997). Control of inflammation, cytokine expression, and germinal center formation by BCL-6. Science (New York, N.Y. 276, 589-592.
Deshmukh, U.S., Bagavant, H., Sim, D., Pidiyar, V., and Fu, S. M. (2007). A SmD peptide induces better antibody responses to other proteins within the small nuclear ribonucleoprotein complex than to SmD protein via intermolecular epitope spreading. J Immunol 178, 2565-2571.
Dienz, O., Eaton, S. M., Bond, J. P., Neveu, W., Moquin, D., Noubade, R., Briso, E. M., Charland, C., Leonard, W. J., Ciliberto, G., et al. (2009). The induction of antibody production by IL-6 is indirectly mediated by IL-21 produced by CD4+ T cells. The Journal of experimental medicine 206, 69-78.
Ettinger, R., Sims, G. P., Fairhurst, A. M., Robbins, R., da Silva, Y. S., Spolski, R., Leonard, W. J., and Lipsky, P. E. (2005). IL-21 induces differentiation of human naive and memory B cells into antibody-secreting plasma cells. J Immunol 175, 7867-7879.
Fazilleau, N., Mark, L., McHeyzer-Williams, L. J., and McHeyzer-Williams, M. G. (2009). Follicular helper T cells: lineage and location. Immunity 30, 324-335.
Feldman, B. M., Rider, L. G., Reed, A. M., and Pachman, L. M. (2008). Juvenile dermatomyositis and other idiopathic inflammatory myopathies of childhood. Lancet 371, 2201-2212.
Forster, R., Emrich, T., Kremmer, E., and Lipp, M. (1994). Expression of the G-protein-coupled receptor BLR1 defines mature, recirculating B cells and a subset of T-helper memory cells. Blood 84, 830-840.
Frentsch, M., Arbach, O., Kirchhoff, D., Moewes, B., Worm, M., Rothe, M., Scheffold, A., and Thiel, A. (2005). Direct access to CD4+ T cells specific for defined antigens according to CD154 expression. Nat Med 11, 1118-1124.
Good, K. L., Bryant, V. L., and Tangye, S. G. (2006). Kinetics of human B cell behavior and amplification of proliferative responses following stimulation with IL-21. J Immunol 177, 5236-5247.
Johnston, R. J., Poholek, A. C., DiToro, D., Yusuf, I., Eto, D., Barnett, B., Dent, A. L., Craft, J., and Crotty, S. (2009). Bcl6 and Blimp-1 are reciprocal and antagonistic regulators of T follicular helper cell differentiation. Science (New York, N.Y. 325, 1006-1010.
Kim, C. H., Lim, H. W., Kim, J. R., Rott, L., Hillsamer, P., and Butcher, E. C. (2004). Unique gene expression program of human germinal center T helper cells. Blood 104, 1952-1960.
Kim, C. H., Rott, L., Kunkel, E. J., Genovese, M. C., Andrew, D. P., Wu, L., and Butcher, E. C. (2001a). Rules of chemokine receptor association with T cell polarization in vivo. The Journal of clinical investigation 108, 1331-1339.
Kim, C. H., Rott, L. S., Clark-Lewis, I., Campbell, D. J., Wu, L., and Butcher, E. C. (2001b). Subspecialization of CXCR5+ T cells: B helper activity is focused in a germinal center-localized subset of CXCR5+ T cells. The Journal of experimental medicine 193, 1373-1381.
King, C., Tangye, S. G., and Mackay, C. R. (2008). T follicular helper (TFH) cells in normal and dysregulated immune responses. Annual review of immunology 26, 741-766.
King, I. L., and Mohrs, M. (2009). IL-4-producing CD4+ T cells in reactive lymph nodes during helminth infection are T follicular helper cells. The Journal of experimental medicine 206, 1001-1007.
Klechevsky, E., Morita, R., Liu, M., Cao, Y., Coquery, S., Thompson-Snipes, L., Briere, F., Chaussabel, D., Zurawski, G., Palucka, A. K., et al. (2008). Functional specializations of human epidermal Langerhans cells and CD14+ dermal dendritic cells. Immunity 29, 497-510.
Kuo, T. C., Shaffer, A. L., Haddad, J., Jr., Choi, Y. S., Staudt, L. M., and Calame, K. (2007). Repression of BCL-6 is required for the formation of human memory B cells in vitro. The Journal of experimental medicine 204, 819-830.
Lane, P. J., Gaspal, F. M., and Kim, M. Y. (2005). Two sides of a cellular coin: CD4(+)CD3− cells regulate memory responses and lymph-node organization. Nat Rev Immunol 5, 655-660.
Lewkowicz, D., and Gottlieb, A. B. (2004). Pediatric psoriasis and psoriatic arthritis. Dermatol Ther 17, 364-375.
Lim, H. W., Lee, J., Hillsamer, P., and Kim, C. H. (2008). Human Th17 cells share major trafficking receptors with both polarized effector T cells and FOXP3+ regulatory T cells J Immunol 180, 122-129.
Linterman, M. A., Beaton, L., Yu, D., Ramiscal, R. R., Srivastava, M., Hogan, J. J., Verma, N. K., Smyth, M. J., Rigby, R. J., and Vinuesa, C. G. (2010). IL-21 acts directly on B cells to regulate Bcl-6 expression and germinal center responses. The Journal of experimental medicine.
Linterman, M. A., Rigby, R. J., Wong, R. K., Yu, D., Brink, R., Cannons, J. L., Schwartzberg, P. L., Cook, M. C., Walters, G. D., and Vinuesa, C. G. (2009). Follicular helper T cells are required for systemic autoimmunity. The Journal of experimental medicine 206, 561-576.
Ma, C. S., Suryani, S., Avery, D. T., Chan, A., Nanan, R., Santner-Nanan, B., Deenick, E. K., and Tangye, S. G. (2009). Early commitment of naive human CD4(+) T cells to the T follicular helper (T(FH)) cell lineage is induced by IL-12. Immunology and cell biology.
MacLennan, I. C. (1994). Germinal centers. Annual review of immunology 12, 117-139.
MacLennan, I. C., Toellner, K. M., Cunningham, A. F., Serre, K., Sze, D. M., Zuniga, E., Cook, M. C., and Vinuesa, C. G. (2003). Extrafollicular antibody responses. Immunological reviews 194, 8-18.
Martin, F., and Chan, A. C. (2006). B cell immunobiology in disease: evolving concepts from the clinic. Annual review of immunology 24, 467-496.
Meffre, E., and Wardemann, H. (2008). B-cell tolerance checkpoints in health and autoimmunity. Current opinion in immunology 20, 632-638.
Mietzner, B., Tsuiji, M., Scheid, J., Velinzon, K., Tiller, T., Abraham, K., Gonzalez, J. B., Pascual, V., Stichweh, D., Wardemann, H., and Nussenzweig, M. C. (2008). Autoreactive IgG memory antibodies in patients with systemic lupus erythematosus arise from nonreactive and polyreactive precursors. Proceedings of the National Academy of Sciences of the United States of America 105, 9727-9732.
Moore, K. W., O'Garra, A., de Waal Malefyt, R., Vieira, P., and Mosmann, T. R. (1993). Interleukin-10. Annual review of immunology 11, 165-190.
Mosmann, T. R., and Coffman, R. L. (1989). TH1 and TH2 cells: different patterns of lymphokine secretion lead to different functional properties. Annual review of immunology 7, 145-173.
Nurieva, R. I., Chung, Y., Hwang, D., Yang, X. O., Kang, H. S., Ma, L., Wang, Y. H., Watowich, S. S., Jetten, A. M., Tian, Q., and Dong, C. (2008). Generation of T follicular helper cells is mediated by interleukin-21 but independent of T helper 1, 2, or 17 cell lineages. Immunity 29, 138-149.
Nurieva, R. I., Chung, Y., Martinez, G. J., Yang, X. O., Tanaka, S., Matskevitch, T. D., Wang, Y. H., and Dong, C. (2009). Bcl6 mediates the development of T follicular helper cells. Science (New York, N.Y. 325, 1001-1005.
Pene, J., Gauchat, J. F., Lecart, S., Drouet, E., Guglielmi, P., Boulay, V., Delwail, A., Foster, D., Lecron, J. C., and Yssel, H. (2004). Cutting edge: IL-21 is a switch factor for the production of IgG1 and IgG3 by human B cells. J Immunol 172, 5154-5157.
Poholek, A. C., Hansen, K., Hernandez, S. G., Eto, D., Chandele, A., Weinstein, J. S., Dong, X., Odegard, J. M., Kaech, S. M., Dent, A. L., et al. (2010). In Vivo Regulation of Bcl6 and T Follicular Helper Cell Development. J Immunol doi:10.4049/jimmunol.0904023.
Rabin, R. L., Alston, M. A., Sircus, J. C., Knollmann-Ritschel, B., Moratz, C., Ngo, D., and Farber, J. M. (2003). CXCR3 is induced early on the pathway of CD4+ T cell differentiation and bridges central and peripheral functions. J Immunol 171, 2812-2824.
Rasheed, A. U., Rahn, H. P., Sallusto, F., Lipp, M., and Muller, G. (2006). Follicular B helper T cell activity is confined to CXCR5(hi)ICOS(hi) CD4 T cells and is independent of CD57 expression. European journal of immunology 36, 1892-1903.
Reinhardt, R. L., Liang, H. E., and Locksley, R. M. (2009). Cytokine-secreting follicular T cells shape the antibody repertoire. Nature immunology 10, 385-393.
Rivino, L., Messi, M., Jarrossay, D., Lanzavecchia, A., Sallusto, F., and Geginat, J. (2004). Chemokine receptor expression identifies Pre-T helper (Th)1, Pre-Th2, and nonpolarized cells among human CD4+ central memory T cells. The Journal of experimental medicine 200, 725-735.
Sallusto, F., Lenig, D., Mackay, C. R., and Lanzavecchia, A. (1998). Flexible programs of chemokine receptor expression on human polarized T helper 1 and 2 lymphocytes. The Journal of experimental medicine 187, 875-883.
Samuels, J., Ng, Y. S., Coupillaud, C., Paget, D., and Meffre, E. (2005). Impaired early B cell tolerance in patients with rheumatoid arthritis. The Journal of experimental medicine 201, 1659-1667.
Schaerli, P., Loetscher, P., and Moser, B. (2001). Cutting edge: induction of follicular homing precedes effector Th cell development. J Immunol 167, 6082-6086.
Schaerli, P., Willimann, K., Lang, A. B., Lipp, M., Loetscher, P., and Moser, B. (2000). CXC chemokine receptor 5 expression defines follicular homing T cells with B cell helper function. The Journal of experimental medicine 192, 1553-1562.
Schmitt, N., Morita, R., Bourdery, L., Bentebibel, S. E., Zurawski, S. M., Banchereau, J., and Ueno, H. (2009). Human dendritic cells induce the differentiation of interleukin-21-producing T follicular helper-like cells through interleukin-12. Immunity 31, 158-169.
Shlomchik, M. J., Marshak-Rothstein, A., Wolfowicz, C. B., Rothstein, T. L., and Weigert, M. G. (1987). The role of clonal selection and somatic mutation in autoimmunity. Nature 328, 805-811.
Simpson, N., Gatenby, P. A., Wilson, A., Malik, S., Fulcher, D. A., Tangye, S. G., Manku, H., Vyse, T. J., Roncador, G., Huttley, G. A., et al. (2010). Expansion of circulating T cells resembling follicular helper T cells is a fixed phenotype that identifies a subset of severe systemic lupus erythematosus. Arthritis and rheumatism 62, 234-244.
Singh, S. P., Zhang, H. H., Foley, J. F., Hedrick, M. N., and Farber, J. M. (2008). Human T cells that are able to produce IL-17 express the chemokine receptor CCR6. J Immunol 180, 214-221.
Song, K., Rabin, R. L., Hill, B. J., De Rosa, S. C., Perfetto, S. P., Zhang, H. H., Foley, J. F., Reiner, J. S., Liu, J., Mattapallil, J. J., et al. (2005). Characterization of subsets of CD4+ memory T cells reveals early branched pathways of T cell differentiation in humans. Proceedings of the National Academy of Sciences of the United States of America 102, 7916-7921.
Suber, T. L., Casciola-Rosen, L., and Rosen, A. (2008). Mechanisms of disease: autoantigens as clues to the pathogenesis of myositis. Nat Clin Pract Rheumatol 4, 201-209.
Subramanian, S., Tus, K., Li, Q. Z., Wang, A., Tian, X. H., Zhou, J., Liang, C., Bartov, G., McDaniel, L. D., Zhou, X. J., et al. (2006). A Tlr7 translocation accelerates systemic autoimmunity in murine lupus. Proceedings of the National Academy of Sciences of the United States of America 103, 9970-9975.
Tsuji, M., Komatsu, N., Kawamoto, S., Suzuki, K., Kanagawa, O., Honjo, T., Hori, S., and Fagarasan, S. (2009). Preferential generation of follicular B helper T cells from Foxp3+ T cells in gut Peyer's patches. Science (New York, N.Y. 323, 1488-1492.
Vinuesa, C. G., Cook, M. C., Angelucci, C., Athanasopoulos, V., Rui, L., Hill, K. M., Yu, D., Domaschenz, H., Whittle, B., Lambe, T., et al. (2005a). A RING-type ubiquitin ligase family member required to repress follicular helper T cells and autoimmunity. Nature 435, 452-458.
Vinuesa, C. G., Tangye, S. G., Moser, B., and Mackay, C. R. (2005b). Follicular B helper T cells in antibody responses and autoimmunity. Nat Rev Immunol 5, 853-865.
Vogelzang, A., McGuire, H. M., Yu, D., Sprent, J., Mackay, C. R., and King, C. (2008). A fundamental role for interleukin-21 in the generation of T follicular helper cells. Immunity 29, 127-137.
Walsh, R. J., Kong, S. W., Yao, Y., Jallal, B., Kiener, P. A., Pinkus, J. L., Beggs, A. H., Amato, A. A., and Greenberg, S. A. (2007). Type I interferon-inducible gene expression in blood is present and reflects disease activity in dermatomyositis and polymyositis. Arthritis and rheumatism 56, 3784-3792.
Ye, B. H., Cattoretti, G., Shen, Q., Zhang, J., Hawe, N., de Waard, R., Leung, C., Nouri-Shirazi, M., Orazi, A., Chaganti, R. S., et al. (1997). The BCL-6 proto-oncogene controls germinal-centre formation and Th2-type inflammation. Nat Genet 16, 161-170.
Yu, D., Rao, S., Tsai, L. M., Lee, S. K., He, Y., Sutcliffe, E. L., Srivastava, M., Linterman, M., Zheng, L., Simpson, N., et al. (2009). The transcriptional repressor Bcl-6 directs T follicular helper cell lineage commitment. Immunity 31, 457-468.
Yurasov, S., Wardemann, H., Hammersen, J., Tsuiji, M., Meffre, E., Pascual, V., and Nussenzweig, M. C. (2005). Defective B cell tolerance checkpoints in systemic lupus erythematosus. The Journal of experimental medicine 201, 703-711.
Zaretsky, A. G., Taylor, J. J., King, I. L., Marshall, F. A., Mohrs, M., and Pearce, E. J. (2009). T follicular helper cells differentiate from Th2 cells in response to helminth antigens. The Journal of experimental medicine 206, 991-999.
Zotos, D., Coquet, J. M., Zhang, Y., Light, A., D'Costa, K., Kallies, A., Corcoran, L. M., Godfrey, D. I., Toellner, K. M., Smyth, M. J., et al. (2010). IL-21 regulates germinal center B cell differentiation and proliferation through a B cell-intrinsic mechanism. The Journal of experimental medicine 207, 365-378.

### REFERENCES

### Example 2

U.S. Pat. No. 7,820,790: IL-6 Binding Proteins.
U.S. Patent Application Publication No. 20080254137: Treatment of Autoimmune Disorders Using Detoxified Cobratoxin.
Breitfeld, D., Ohl, L., Kremmer, E., Ellwart, J., Sallusto, F., Lipp, M., and Forster, R. (2000). Follicular B helper T cells express CXC chemokine receptor 5, localize to B cell follicles, and support immunoglobulin production. The Journal of experimental medicine 192, 1545-1552.
Fazilleau, N., Mark, L., McHeyzer-Williams, L. J., and McHeyzer-Williams, M. G. (2009). Follicular helper T cells: lineage and location. Immunity 30, 324-335.
Johnston, R. J., Poholek, A. C., DiToro, D., Yusuf, I., Eto, D., Barnett, B., Dent, A. L., Craft, J., and Crotty, S. (2009). Bcl6 and Blimp-1 are reciprocal and antagonistic regulators of T follicular helper cell differentiation. Science (New York, N.Y. 325, 1006-1010.
Kim, C. H., Rott, L. S., Clark-Lewis, I., Campbell, D. J., Wu, L., and Butcher, E. C. (2001). Subspecialization of CXCR5+ T cells: B helper activity is focused in a germinal center-localized subset of CXCR5+ T cells. The Journal of experimental medicine 193, 1373-1381.
King, C., Tangye, S. G., and Mackay, C. R. (2008). T follicular helper (TFH) cells in normal and dysregulated immune responses. Annual review of immunology 26, 741-766.
Nurieva, R. I., Chung, Y., Martinez, G. J., Yang, X. O., Tanaka, S., Matskevitch, T. D., Wang, Y. H., and Dong, C. (2009). Bcl6 mediates the development of T follicular helper cells. Science (New York, N.Y. 325, 1001-1005.
Schaerli, P., Willimann, K., Lang, A. B., Lipp, M., Loetscher, P., and Moser, B. (2000). CXC chemokine receptor 5 expression defines follicular homing T cells with B cell helper function. The Journal of experimental medicine 192, 1553-1562.
Schmitt, N., Morita, R., Bourdery, L., Bentebibel, S. E., Zurawski, S. M., Banchereau, J., and Ueno, H. (2009). Human dendritic cells induce the differentiation of interleukin-21-producing T follicular helper-like cells through interleukin-12. Immunity 31, 158-169.
Vinuesa, C. G., Tangye, S. G., Moser, B., and Mackay, C. R. (2005). Follicular B helper T cells in antibody responses and autoimmunity. Nat Rev Immunol 5, 853-865.
Yu, D., Rao, S., Tsai, L. M., Lee, S. K., He, Y., Sutcliffe, E. L., Srivastava, M., Linterman, M., Zheng, L., Simpson, N., et al. (2009). The transcriptional repressor Bcl-6 directs T follicular helper cell lineage commitment. Immunity 31, 457-468.

What is claimed is:

1. A method for treating dermatomyositis comprising the steps of:
identifying a subject in need of treatment for dermatomyositis;
isolating one or more T cells from the subject;
isolating and purifying one or more CD4⁺X3⁻R6⁻ T cells or CD4⁺X3⁻R6⁺ T cells from the one or more T cells;
activating the CD4⁺X3⁻R6⁻ T cells or CD4⁺X3⁻R6⁺ T cells expand the Type 1 T cells; and
providing the one or more isolated and purified CD4⁺X3⁻R6⁻ T cells or CD4⁺X3⁻R6⁺ T cells to the subject.

2. A method for providing protective mucosal immunity in a subject comprising the steps of:
identifying a subject in need of protective mucosal immunity;
isolating one or more T cells from the subject;
isolating and purifying one or more CD4⁺X3⁻R6⁺ T cells from the one or more T cells; and
providing the one or more isolated and purified CD4⁺ X3⁻R6⁺ T cells to the subject prior to vaccination to induce Type 17 cells.

3. A method for inducing the differentiation of naïve B cells towards plasmablasts in a subject comprising:
providing one or more isolated and purified CD4⁺ T cells selected from X5⁺ CD4⁺ T cells or X5⁻ CD4⁺ T cells to a subject.

4. The method of claim 3, wherein the one or more isolated and purified CD4⁺ T cells are X5⁺ CD4⁺ X3⁻R6⁻ T cells or X5⁻ CD4⁺ X3⁻R6⁻ T cells that secrete IL-4, IL-5, and IL-13.

5. The method of claim 3, wherein the one or more isolated and purified CD4⁺ T cells are X5⁺ CD4⁺ X3⁻R6⁺ T cells or X5⁻ CD4⁺ X3⁻R6⁺ T cells that secrete IL-17A and IL-22

6. The method of claim 3, wherein the one or more isolated and purified CD4⁺ T cells are Th17 cells that secrete IL-17A and IL-22.

7. The method of claim 3, wherein the CD4⁺ T cells comprise X5⁻ CD4⁺X3⁺ R6⁺ T cells, X5⁻ CD4⁺X3⁻R6⁻ T cells, or X5⁻ CD4⁺ X3⁻R6⁺ T cells.

8. The method of claim 3, wherein the CD4⁺ T cells are X5⁻ CD4⁺ T cell that induce naïve B cells to produce IFN-γ, IL-22, T-bet and RORγT.

9. The method of claim 3, wherein the CD4⁺ T cells are X5⁺ CD4⁺ T cell comprising Th1 T cells, Th2 T cells, or Th17 T cells.

10. The method of claim 9, wherein the Th1 T cells induce naïve B cells to produce T-bet.

11. The method of claim 9, wherein the Th2 T cells induce naïve B cells to produce one or more Igs, GATA3 or a combination thereof.

12. The method of claim 9, wherein the Th2 T cells induce naïve B cells to produce IgM, IgG, IgA, IgE or a combination thereof.

13. The method of claim 9, wherein the Th2 T cells induce naïve B cells to produce IL4, IL5, IL13, IL21 or a combination thereof.

14. The method of claim 9, wherein the Th17 T cells induce naïve B cells to produce ROR γ T.

15. The method of claim 9, wherein the Th17 T cells induce naïve B cells to produce IL17A, IL22 or a combination thereof.

16. The method of claim 9, wherein the Th17 T cells induce naïve B cells to produce IgM, and isotype switching towards IgG and IgA.

17. The method of claim 3 wherein the CD4⁺ T cells are X5⁻ CD4⁺ T cell comprising Th1 T cells, Th2 T cells, Th17 T cells or a combination thereof.

18. The method of claim 9, wherein the Th2 T cells induce naïve B cells to produce IgM, and IgE.

19. An isolated and purified CD4⁺ T cell comprising:
one or more isolated and purified CD4⁺ T cells selected from one or more isolated and purified X5⁺CD4⁺ T cells or one or more isolated and purified X5⁻CD4⁺ T cells.

20. The isolated and purified T cell of claim 19, wherein the one or more isolated and purified CD4⁺ T cells are X5⁺ CD4⁺ X3⁻R6⁻ T cells secrete IL-4, IL-5, and IL-13.

21. The isolated and purified T cell of claim 19, wherein the one or more isolated and purified CD4⁺ T cells are X5⁺ CD4⁺X3⁻R6⁺ T cells or X5⁻ CD4⁺X3⁻R6⁺ T cells that secrete IL-17A and IL-22.

22. The isolated and purified T cell of claim 19, wherein the one or more isolated and purified CD4⁺ T cells are X5^{±} CD4⁺ X3⁺R6⁺ T cells, X5^{±} CD4⁺ X3⁻R6⁻ T cells, X5^{±} CD4⁺ X3⁻ R6⁺ T cells or a combination thereof.

23. The isolated and purified T cell of claim 19, wherein the one or more isolated and purified X5⁻CD4⁺ T cells induce naïve B cells to produce IFN-γ, IL-22, T-bet and RORγT.

24. The isolated and purified T cell of claim 19, wherein the one or more isolated and purified X5^{±} CD4⁺ T cells are Th17 cells that secrete IL-17A and IL-22.

25. The isolated and purified T cell of claim 19, wherein the one or more X5⁺CD4⁺ T cell comprise Th1 T cells, Th2 T cells, or Th17 T cells.

26. The isolated and purified T cell of claim 25, wherein the Th1 T cells induce naïve B cells to produce T-bet.

27. The isolated and purified T cell of claim 25, wherein the Th2 T cells induce naïve B cells to produce Igs, GATA3 or a combination thereof.

28. The isolated and purified T cell of claim 25, wherein the Th2 T cells induce naïve B cells to produce IgM, IgG, IgA, IgE or a combination thereof.

29. The isolated and purified T cell of claim 25, wherein the Th2 T cells induce naïve B cells to produce IL4, IL5, IL13, IL21 or a combination thereof.

30. The isolated and purified T cell of claim 25, wherein the Th17 T cells induce naïve B cells to produce ROR γ T.

31. The isolated and purified T cell of claim 25, wherein the Th17 T cells induce naïve B cells to produce IL17A and IL22.

32. The isolated and purified T cell of claim 25, wherein the Th17 T cells induce naïve B cells to produce IgM, and isotype switching towards IgG and IgA.

33. The isolated and purified T cell of claim 19, wherein the one or more X5⁻ CD4⁺ T cell comprise Th1 T cells, Th2 T cells, or Th17 T cells.

34. The isolated and purified T cell of claim 33, wherein the Th2 T cells induce naïve B cells to produce IgM, and IgE.

35. The isolated and purified T cell of claim 33, wherein the Th2 T cells induce naïve B cells to produce IL4, IL5, IL13, IL21 or a combination thereof.

36. The isolated and purified T cell of claim 33, wherein the Th17 T cells induce naïve B cells to produce IL17A and IL22.

37. A method for increasing the effectiveness of antigen presentation in a subject comprising the steps of:
identifying a subject in need of treatment;
isolating one or more T cells from the subject;
isolating and purifying one or more X5 CD4⁺ T cells from the one or more T cells; and
providing the one or more isolated and purified X5 CD4⁺ T cells to the subject.

38. A method for the modulation of cytokine secretion comprising the steps of:
contacting a naïve B cells with one or more CXCR5⁺ CD4⁺ T cell to produce one or more cytokines.

39. A method for affecting functional differences of blood memory CD4+ T cell populations in a subject comprising the steps of:
identifying a subject in need of treatment;
isolating and purifying one or more X5⁺ CD4⁺ T cells or X5⁻ CD4⁺ T cells from the subject; and
providing the one or more X5⁺ CD4⁺ T cells or one or more X5⁻ CD4⁺ T cells to a subject.

40. A method for the modulation of systemic autoimmunity comprising the steps of:
identifying a subject in need of treatment;
isolating and purifying one or more X5⁺ CD4⁺ T cells or X5⁻ CD4⁺ T cells from the subject; and
providing the one or more X5⁺ CD4⁺ T cells or one or more X5⁻ CD4⁺ T cells to a subject.

41. An pharmaceutical composition for the modulation of systemic autoimmunity comprising:
a pharmaceutically acceptable carrier containing a pharmaceutically acceptable amount of one or more isolated and purified X5⁺CD4⁺ Th1 T cells, X5⁺CD4⁺ Th2 T cells, X5⁺CD4⁺ Th17 T cells, X5⁻CD4⁺ Th1 T cells, X5⁻CD4⁺ Th2 T cells, or X5⁻CD4⁺ Th17 T cells.

42. A pharmaceutical composition for the treatment of juvenile dermatomyositis comprising:
a pharmaceutically acceptable carrier containing a pharmaceutically acceptable amount of one or more isolated and purified X5⁺CD4⁺ Th1 T cells, X5⁺CD4⁺ Th2 T cells, X5⁺CD4⁺ Th17 T cells, X5⁻CD4⁺ Th1 T cells, X5⁻CD4⁺ Th2 T cells, or X5⁻CD4⁺ Th17 T cells.

43. A method for promoting development of IL-21 producing T follicular helper cells (Tfh) in a subject from naïve CD4⁺ T cells comprising the steps of:
providing the one or more naïve CD4⁺ T cells;
contacting the one or more naïve CD4⁺ T cells with a cytokine or a cytokine cocktail selected from:
IL-6/TGF-b/IL-12; or
IL-12; or
TGF-b and IL-12; and
differentiating the one or more naïve CD4⁺ T cells into the one or more IL-21 producing Tfh cells.

44. The method of claim 43, wherein the one or more activated CD4+ T cells are CXCR5+CD4+X3+ R6+ T cells, CXCR5+ CD4+X3−R6− T cells, CXCR5+ CD4+X3−R6+ T cells, or a combination thereof.

45. The method of claim 43, further comprising the step of activating the one or more Tfh cells with anti-CD3/CD28 mAbs.

46. The method of claim 43, wherein the one or more activated CD4+ T cells produce Ig's, GATA3, or a combination thereof.

47. The method of claim 43, wherein the one or more activated CD4+ T cells produce IgM, IgG, IgA, IgE, or a combination thereof.

48. A method for regulating or suppressing development of one or more Th1 cells by naïve CD4⁺ T cells in a subject comprising the steps of:
isolating and purifying the one or more naïve CD4⁺ T cells from subject;
contacting the one or more naïve CD4⁺ T cells with a cytokine cocktail comprising IL-6/TGF-b/IL-12 to suppress the development of one or more Th1 cells by the naïve CD4+ T cells; and
modifying the expression of one or more factors from the one or more naïve CD4⁺ T cells.

49. The method of claim 48, wherein the method results in a modification of an expression of one or more factors by the naïve CD4⁺ T cells, wherein the modification comprises an increased expression of IL-21, a decreased expression of IFN-g, an increased expression of CXCR5, an increased expression of Bcl-6, a decreased expression of Blimp-1, or a combination thereof.

50. A method for promoting the development of T follicular helper (Tfh) cells from naïve CD4+ T cells, modifying expression of one or more factors by the naïve CD4+ T cells, or both in a subject comprising the steps of:
isolating and purifying one or more naïve CD4⁺ T cells from subject;
promoting differentiation of the one or more naïve CD4⁺ T cells to one or more Tfh cells by contacting the one or more naïve CD4⁺ T cells with a cytokine or a cytokine cocktail, wherein the cytokine or cytokine cocktail is selected from:
IL-6 and TGF-b and IL-12; or
IL-12; or
TGF-b and IL-12; and
activating the one or more one or more Tfh cells with an anti-CD3/CD28 mAbs to modify the expression of one or more factors, wherein the modification comprises an increase in an expression of IL-21, a decrease in the expression of IFN-g, an increase in the expression of CXCR5, an increase in the expression of Bcl-6, a decrease in the expression of Blimp-1, or a combination thereof.

51. The method of claim 49, wherein the one or more naïve CD4+ T cells are activated for at least 1, 2, 3, 4, 5, 6, 7, 8, 9, 10, 11, or more days.

52. The method of claim 51, wherein the one or more naïve CD4+ T cells are activated for at least 7 days.

53. The method of claim 49, further comprising the step of re-stimulation with PMA/ionomycin.

54. A method for promoting the development of T follicular helper (Tfh) cells in a subject comprising the steps of:
isolating and purifying one or more naïve CD4⁺ T cells from subject;
contacting the one or more naïve CD4⁺ T cells with Bcl-6; and
promoting the development of the one or more naïve CD4⁺ T cells into one or more Tfh cells.

55. The method of claim 54, further comprising the step of activating the one or more one or more Tfh cells with anti-CD3/CD28 mAbs.

56. A method for promoting the differentiation of CD4+ T cells into T follicular helper (Tfh) cells in a subject comprising the steps of:
providing one or more CD4⁺ T cells;
contacting the one or more CD4⁺ T cells with one or more cytokines selected from IL-6, IL-12 and TGF-b; and
differentiating the one or more CD4⁺ T cells into one or more Tfh cells.

57. The method of claim 56, further comprising the step of activating the one or more Tfh cells with anti-CD3/CD28 mAbs, wherein the one or more Tfh cells produce a specific antibody response.

58. The method of claim 56, wherein the subject is in need of immunostimulation or an enhanced immune response.

59. A method for suppressing development of one or more T follicular helper (Tfh) cells in a subject comprising the steps of:
identifying the subject in need of suppression of the immune response by reduction in one or more Tfh cells; and
administering a therapeutically effective amount of an IL-6 antagonist, an IL-6 inhibitor, an anti IL-6 agent, or any combinations thereof to the subject in an amount sufficient to reduce differentiation of CD4⁺ T cells into one or more Tfh cells.

60. The method of claim 59, wherein the IL-6 antagonist, the IL-6 inhibitor, the anti IL-6 agent, or any combinations thereof comprise lunasin, tocilizumab, sirukumab, Elsilimomab, an anti-IL-6 monoclonal antibody, 20S,21-epoxy-resibufogenin-3-formate (ERBF), or any combinations thereof.

61. A method for suppressing the expression of IFN-g in a subject comprising the step of stimulating naïve CD4+ T cells with IL-6/TGF-b/IL-12, wherein the level of IFN-g expressed is less than that stimulated with IL-12 alone or a combination of IL-12/TGF-b.

62. The method of claim 61, wherein the naïve CD4+ T cells express IL-21 but not IFN-g.

63. A method for stimulating chemokine receptor expression levels of T follicular helper (Tfh) cells in a subject comprising the steps of:
providing one or more naïve CD4+ T cells;
contacting the one or more naïve CD4+ T cells with IL-6/TGF-b/IL-12; and
increasing the expression of a chemokine receptor in a human Tfh cell, wherein the level of levels of CXCR5 is higher than the level of CXCR5 when stimulated with IL-12 alone and the chemokine receptor plays a central role for their migration into B cell follicles.

64. A method for treating an autoimmune disease in a subject comprising the steps of:
identifying the subject in need of treatment against the autoimmune disease; and
administering to the subject a therapeutically effective amount of a composition comprising an IL-6 antagonist, an IL-6 inhibitor, an anti IL-6 agent, or any combinations thereof to the subject in an amount sufficient to treat the autoimmune disease, wherein the composition treats the autoimmune disease by reducing a differentiation of CD4+ T cells in the human subject into one or more T follicular helper (Tfh) cells.

65. The method of claim 64, wherein the IL-6 antagonist, the IL-6 inhibitor, the anti IL-6 agent, or any combinations thereof comprise lunasin, tocilizumab, sirukumab, Elsilimomab, an anti-IL-6 monoclonal antibody, 20S,21-epoxy-resibufogenin-3-formate (ERBF), or any combinations thereof.

66. The method of claim 64, wherein the autoimmune disease is dermatomyositis.

67. A IL-6 activated CD4⁺ T cell comprising one or more isolated and purified activated CD4⁺ T cell that expresses IL-21, CXCR5, and Bcl-6 and does not express IFN-g or Blimp-1 as a result of contact with IL-6/IL-12/TGF-b to induce the CD4+ T cells to express IL-21, CXCR5, and Bcl-6 and to acquire the capacity to help B cells.

68. An pharmaceutical composition for the modulation of systemic autoimmunity comprising a pharmaceutically acceptable carrier containing a pharmaceutically acceptable amount of one or more isolated and purified activated CD4⁺ T cell that expresses IL-21, CXCR5, and Bcl-6 and does not express IFN-g or Blimp-1 as a result of contact with IL-6/IL-12/TGF-b to induce the CD4+ T cells to express IL-21, CXCR5, and Bcl-6 and to acquire the capacity to help B cells, wherein the one or more isolated and purified activated CD4⁺ T cell are CXCR5⁺CD4⁺ Th1 T cells, CXCR5⁺CD4⁺ Th2 T cells, CXCR5⁺CD4⁺ Th17 T cells, CXCR5⁻CD4⁺ Th1 T cells, CXCR5⁻CD4⁺ Th2 T cells, or CXCR5⁻CD4⁺ Th17 T cells.

69. A method for enhancing the migration of T follicular helper (Tfh) cells into B cell follicles in a subject comprising the steps of:
identifying the subject in need of enhanced migration of the one or more Tfh cells into B cell follicules;
isolating and purifying one or more CD4⁺ T cells from the subject;
activating the one or more CD4⁺ T cells with IL-6/IL-12/TGF-b to form one or more activated CD4⁺ T cells that express IL-21, CXCR5 and Bcl-6, and that acquire increased capacity to migrate into B cells to help B cells; and
reintroducing the one or more activated CD4⁺ T cells into the subject.

70. A method for stimulating IgG production in a subject comprising the steps of:
isolating one or more T cells from the subject;
isolating and purifying one or more CD4⁺ T cells from the one or more T cells;
activating the one or more CD4⁺ T cells with IL-6/IL-12/TGF-b to form one or more activated CD4⁺ T cells that express IgG, wherein the levels of IgG is higher than the level of IgG stimulated with IL-12 alone or the combination of IL-12/TGF-b; and
reintroducing the one or more activated CD4⁺ T cells into the subject.

71. A method for sending one or more activation signals through STAT3 in a subject comprising the steps of:
isolating and purifying one or more CD4⁺ T cells from the subject;
activating the one or more CD4⁺ T cells with IL-6/IL-12/TGF-b to form one or more activated CD4⁺ T cells that differentiate into one or more T follicular helper (Tfh) cells; and
reintroducing the one or more activated CD4⁺ T cells into the subject.


SEQUENCE LISTING (14 sequences)

SEQ ID NO: 1 - DNA, 24 bp, artificial sequence
  synthetic oligonucleotide
cactacaggatgtttgtggacgtg

SEQ ID NO: 2 - DNA, 24 bp, artificial sequence
  synthetic oligonucleotide
ccccttgttgtttgtgagctttag

SEQ ID NO: 3 - DNA, 19 bp, artificial sequence
  synthetic oligonucleotide
tgtctgcagccaggagagc

SEQ ID NO: 4 - DNA, 22 bp, artificial sequence
  synthetic oligonucleotide
atgcatcaaacaactgtggcca

SEQ ID NO: 5 - DNA, 24 bp, artificial sequence
  synthetic oligonucleotide
tctggagctggcctttcatcatca

SEQ ID NO: 6 - DNA, 24 bp, artificial sequence
  synthetic oligonucleotide
tctgctcacttccaaagagctggt

SEQ ID NO: 7 - DNA, 21 bp, artificial sequence
  synthetic oligonucleotide
tcctgtggcatccacgaaact

SEQ ID NO: 8 - DNA, 21 bp, artificial sequence
  synthetic oligonucleotide
gaagcatttgcggtggacgat

SEQ ID NO: 9 - DNA, 18 bp, artificial sequence
  synthetic oligonucleotide
ttccgctacaagggcaac

SEQ ID NO: 10 - DNA, 20 bp, artificial sequence
  synthetic oligonucleotide
tgcaacgatagggtttctca

SEQ ID NO: 11 - DNA, 20 bp, artificial sequence
  synthetic oligonucleotide
gtggtgggttaatcggtttg

SEQ ID NO: 12 - DNA, 21 bp, artificial sequence
  synthetic oligonucleotide
gaagctcccctctggaataga

SEQ ID NO: 13 - DNA, 25 bp, artificial sequence
  synthetic oligonucleotide
gactttggttatcttcgcaataaga

SEQ ID NO: 14 - DNA, 20 bp, artificial sequence
  synthetic oligonucleotide
aggtcccagtccgagatgta